SEQUENCE LISTING (20 sequences)

SEQ ID NO: 1 - protein, 87 aa, SARS-CoV-2
LQSYGFQPTNGVGYQPYRVVVLSFELLHAPATVCGPKKSTNLVKNKCVNFNFNGLTGTGVLTESNKKFLPFQQFGRDIADTTDAVRD

SEQ ID NO: 2 - protein, 31 aa, SARS-CoV-2
LPDPSKPSKRSFIEDLLFNKVTLADAGFIKQ

SEQ ID NO: 3 - protein, 80 aa, SARS-CoV-2
NFRVVPSGDVVRFPNITNLCPFGEVFNATKFPSVYAWERKKISNCVADYSVLYNSTFFSTFKCYGVSATKLNDLCFSNVY

SEQ ID NO: 4 - protein, 80 aa, SARS-CoV-2
NFRVQPTESIVRFPNITNLCPFGEVFNATRFASVYAWNRKRISNCVADYSVLYNSASFSTFKCYGVSPTKLNDLCFTNVY

SEQ ID NO: 5 - protein, 80 aa, SARS-CoV-2
ADSFVVKGDDVRQIAPGQTGVIADYNYKLPDDFMGCVLAWNTRNIDATSTGNYNYKYRYLRHGKLRPFERDISNVPFSPD

SEQ ID NO: 6 - protein, 80 aa, SARS-CoV-2
ADSFVIRGDEVRQIAPGQTGKIADYNYKLPDDFTGCVIAWNSNNLDSKVGGNYNYLYRLFRKSNLKPFERDISTEIYQAG

SEQ ID NO: 7 - protein, 79 aa, SARS-CoV-2
GKPCTPPALNCYWPLNDYGFYTTTGIGYQPYRVVVLSFELLNAPATVCGPKLSTDLIKNQCVNFNFNGLTGTGVLTPSS

SEQ ID NO: 8 - protein, 80 aa, SARS-CoV-2
STPCNGVEGFNCYFPLQSYGFQPTNGVGYQPYRVVVLSFELLHAPATVCGPKKSTNLVKNKCVNFNFNGLTGTGVLTESN

SEQ ID NO: 9 - protein, 80 aa, SARS-CoV-2
KRFQPFQQFGRDVSDFTDSVRDPKTSEILDISPCAFGGVSVITPGTNASSEVAVLYQDVNCTDVSTAIHADQLTPAWRIY

SEQ ID NO: 10 - protein, 80 aa, SARS-CoV-2
KKFLPFQQFGRDIADTTDAVRDPQTLEILDITPCSFGGVSVITPGTNTSNQVAVLYQDVNCTEVPVAIHADQLTPTWRVY

SEQ ID NO: 11 - protein, 76 aa, SARS-CoV-2
STGNNVFQTQAGCLIGAEHVDTSYECDIPIGAGICASYHTVSLLRSTSQKSIVAYTMSLGADSSIAYSNNTIAIPT

SEQ ID NO: 12 - protein, 70 aa, SARS-CoV-2
STGSNVFQTRAGCLIGAEHVNNSYCASYQTQTNSPRRARSVASQSIIAYTMSLGAENSVAYSNNSIAIPT

SEQ ID NO: 13 - protein, 80 aa, SARS-CoV-2
NFSISITTEVMPVSMAKTSVDCNMYICGDSTECANLLLQYGSFCTQLNRALSGIAAEQDRNTREVFAQVKQMYKTPTLKY

SEQ ID NO: 14 - protein, 70 aa, SARS-CoV-2
NFTISVTTEILPVSMTKTSVDCTMYICGDSTECSNLLLQYGSFCTQLNRALTGIAVEQDKNTQETPPIKD

SEQ ID NO: 15 - protein, 80 aa, SARS-CoV-2
FGGFNFSQILPDPLKPTKRSFIEDLLFNKVTLADAGFMKQYGECLGDINARDLICAQKFNGLTVLPPLLTDDMIAAYTAA

SEQ ID NO: 16 - protein, 80 aa, SARS-CoV-2
FGGFNFSQILPDPSKPSKRSFIEDLLFNKVTLADAGFIKQYGDCLGDIAARDLICAQKFNGLTVLPPLLTDEMIAQYTSA

SEQ ID NO: 17 - protein, 18 aa, SARS-CoV-2
TESNKKFLPFQQFGRDIA

SEQ ID NO: 18 - protein, 18 aa, SARS-CoV-2
PSKPSKRSFIEDLLFNKV

SEQ ID NO: 19 - protein, 22 aa, SARS-CoV-2
CGGGTESNKKFLPFQQFGRDIA

SEQ ID NO: 20 - protein, 22 aa, SARS-CoV-2
CGGGPSKPSKRSFIEDLLFNKV

(19) United States
(12) Patent Application Publication
(10) Pub. No.: US 2021/0364516 A1
(43) Pub. Date: Nov. 25, 2021

(54) ULTRA-SENSITIVE, FAST, PLASMONIC-FLUOR ENHANCED ASSAY FOR SARS-COV-2 IMMUNE RESPONSE

(71) Applicant: Washington University, St. Louis, MO (US)

(72) Inventors: Jeremiah Morrissey, St. Louis, MO (US); Srikanth Singamaneni, St. Louis, MO (US); Zheyu Wang, St. Louis, MO (US)

(21) Appl. No.: 17/303,063

(22) Filed: May 19, 2021

Related U.S. Application Data
(60) Provisional application No. 63/093,404, filed on Oct. 19, 2020.
(60) Provisional application No. 63/027,178, filed on May 19, 2020.

(51) Int. Cl. G01N 33/569; G01N 21/64

(52) U.S. Cl. CPC G01N 33/56983; G01N 21/6428; G01N 2021/6439; G01N 2800/26; G01N 2333/165

(57) ABSTRACT

The present disclosure is directed to assays for detecting at least one of a SARS-CoV-2 specific antibody, a SARS-COV-2 specific immunoglobulin, and a monoclonal response to single linear neutralizing epitopes within SARS-CoV-2 spike protein, wherein the assay includes a plasmonic-fluor. The assays include multiplexed and ultrafast assays.

### CROSS-REFERENCE TO RELATED APPLICATIONS

This application claims priority to U.S. Provisional Application No. 63/027,178, filed May 19, 2020, and to U.S. Provisional Application No. 63/093,404, filed Oct. 19, 2020, the contents of which are incorporated herein by reference in their entireties.

### STATEMENT REGARDING FEDERALLY SPONSORED RESEARCH & DEVELOPMENT

This invention was made with government support under CA141521 awarded by the National Institutes of Health and under CBET 2027145 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 27, 2021, is named 019439-Sequence-Listing.TXT and is 12,679 bytes in size.

### BACKGROUND OF THE DISCLOSURE

Severe acute respiratory syndrome coronavirus-2 (SARS-CoV-2) has been unprecedentedly threatening the public health worldwide. As of March 2021, more than 119 million cases of coronavirus disease 2019 (COVID-19) have been reported, resulting in over 2.6 million deaths. Although the fast development and administration of vaccines have mitigated the pandemic, it remains important to achieve early diagnosis and improve treatment of COVID-19. Moreover, rapidly spreading SARS-CoV-2 variants have emerged as one of the new challenges as they may jeopardize the efficacy of vaccines and current monoclonal neutralizing antibodies introduced for prototype SARS-CoV-2. Stepping into the post-pandemic era, there is a dire need for novel technologies, which will rapidly and precisely diagnose symptomatic and asymptomatic disease, and predict the infection course, reducing the mortality of COVID-19 patients, as well as evaluate the persistence of acquired immunity against prototypical SARS-CoV-2 and its variants upon vaccination.

Conventional single-plex serology assays employ pristine SARS-CoV-2 spike (S) protein, the receptor binding domain (RBD) of the spike protein or nucleocapsid (N) protein as recognition elements (i.e. as antigen baits) to capture target antibodies. Despite its simplicity and low cost, conventional serological tests only provide coarse information about viral exposure history, infection stage and are variable in predicting neutralizing activity. This limitation primarily stems from the use of whole proteins or even regions of proteins such as the 223 amino acid RBD or the 301 amino acid N-terminal domain (NTD) of the S protein as baits. Detection and quantification of antibodies that bind to specific epitopes within a whole protein or a domain within the whole protein requires highly sensitive detection modalities. With deeper understanding of humoral response, recent studies have discovered that antibodies towards different epitopes may exhibit polarized functions, while part of them will neutralize the interactions between virus and host cells, others may inversely exacerbate patient outcome due to the antibody-dependent enhancement (ADE) effect, correlating with the severity of COVID-19. Therefore, early detection and identification of antibodies targeting precise epitopes will improve the diagnosis and help determine the future protection afforded to patients suffering from mild to severe COVID-19. More importantly, this information can be employed to evaluate and predict the clinical efficacy of vaccines against SARS-CoV-2 including both prototype and variants.

Additionally, as the response to SARS-CoV-2, immune system produces specific IgM upon the infection, while IgG/IgA are produced subsequently following the class switch recombination of B cells. However, IgA has a very important role in the adaptive humoral immune protection at mucosal surfaces, particularly in the respiratory system since the major infection route of SARS-CoV-2 is through the mucosa of the respiratory system. Therefore, measurement of IgA is important in the overall understanding of COVID-19. Further, each antibody isotype has subclasses, which may influence the clinical course of patients with COVID-19. Simultaneous detection of multiple antibody isotypes have been reported to improve the accuracy of diagnostic assay and could provide temporal information on the infection course. However, due to the limited sensitivity, the majority of commercialized assay or previous studies employ pristine virus proteins as antigens or only detect IgG/IgM towards the specific epitopes.

Accordingly, there is a need for rapid, sensitive, and accurate biosensors and assays for COVID-19 that can be broadly deployed to rapidly assess the epidemiology of the disease and limit its outbreak. The embodiments described herein resolve at least these known deficiencies.

### BRIEF DESCRIPTION OF THE DISCLOSURE

In one aspect, the present disclosure is directed to an assay for detecting at least one of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.

In some embodiments, the SARS-CoV-2 specific antibody is selected from IgG and IgM; the SARS-COV-2 specific immunoglobulin is IgA immunoglobulin that recognizes at least one of SARS-CoV-2 nucleocapsid protein, SARS-CoV-2 Spike protein 1, SARS-CoV-2 Spike protein 2, and Receptor Binding Domain of SARS-CoV-2 Spike protein 1; the assay comprises at least one of an immunomicroarray, an enzyme-linked immunoabsorbent assay (ELISA), a fluorescence linked immunosorbent assay (FLISA), bead-based fluoroimmunoassays, and flow cytometry; the monoclonal response to single linear neutralizing epitopes within SARS-CoV-2 spike protein comprises a single well ELISA assay; the monoclonal response to single linear neutralizing epitopes within SARS-CoV-2 spike protein comprises a multiplexed well ELISA assay; the assay is an ultrafast assay; the assay further comprises biotin and streptavidin and/or the assay is a dual-modal colorimetric and fluorescence assay.

In another aspect, the present disclosure is directed to a multiplexed lateral flow assay for detection of two or more of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.

In some embodiments, the SARS-CoV-2 specific antibody is selected from IgG and IgM; the SARS-COV-2 specific immunoglobulin is IgA immunoglobulin that recognizes at least one of SARS-CoV-2 nucleocapsid protein, SARS-CoV-2 Spike protein 1, SARS-CoV-2 Spike protein 2, and Receptor Binding Domain of SARS-CoV-2 Spike protein 1; the assay comprises at least one of an immunomicroarray, an enzyme-linked immunoabsorbent assay (ELISA), a fluorescence linked immunosorbent assay (FLISA), bead-based fluoroimmunoassays, and flow cytometry; the monoclonal response to single linear neutralizing epitopes within SARS-CoV-2 spike protein comprises a single well ELISA assay; the monoclonal response to single linear neutralizing epitopes within SARS-CoV-2 spike protein comprises a multiplexed well ELISA assay; the assay is an ultrafast assay; the assay further comprises biotin and streptavidin and/or the assay is a dual-modal colorimetric and fluorescence assay.

In yet another aspect, the present disclosure is directed to a method for amplification of a polyclonal response to SARS-CoV-2 infection, the method comprising performing an assay for detecting at least one of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments described herein may be better understood by referring to the following description in conjunction with the accompanying drawings.

FIG. 1A is an exemplary embodiment of a schematic illustration depicting the structure of plasmonic-fluor in accordance with the present disclosure. FIG. 1B is an exemplary embodiment of TEM images of bare AuNR and plasmonic-fluor in accordance with the present disclosure. FIG. 1C is an exemplary embodiment of fluorescence intensity of conventional fluor and plasmonic-fluor at different molar concentrations in accordance with the present disclosure. FIG. 1D is an exemplary embodiment of a plot showing IL-6 dose-dependent fluorescence intensity from conventional FLISA as well as corresponding fluorescence intensity map digital photograph in accordance with the present disclosure. FIG. 1E is an exemplary embodiment of a plot showing IL-6 dose-dependent fluorescence intensity from p-FLISA as well as corresponding fluorescence intensity map digital photograph in accordance with the present disclosure. FIG. 1F is an exemplary embodiment of a plot showing IL-6 dose-dependent fluorescence intensity from ELISA (optical density at 450 nm) as well as corresponding fluorescence intensity map digital photograph in accordance with the present disclosure.

FIG. 2A is an exemplary embodiment of dose-response curves of 280-min ELISA in accordance with the present disclosure. FIG. 2B is an exemplary embodiment of dose-response curves of 20-min p-FLISA in accordance with the present disclosure. FIG. 2C is an exemplary embodiment of NGAL concentration in kidney disease and healthy individuals tested by ELISA and p-FLISA in accordance with the present disclosure.

FIG. 3A is an exemplary embodiment of fluorescent dual-modal detection using plasmonic-fluor in protein arrays in accordance with the present disclosure. FIG. 3B is an exemplary embodiment of colorimetric dual-modal detection using plasmonic-fluor in protein arrays in accordance with the present disclosure.

FIG. 4 is an exemplary embodiment of a schematic illustration of plasmonically-enhanced rapid detection of corona virus (COVID-19) in accordance with the present disclosure.

FIG. 5 is an exemplary embodiment of interpreting diagnostic tests for SARS-CoV-2 and estimated variation over time in diagnostic tests for detection of SARS-CoV-2 infection relative to symptom onset in accordance with the present disclosure. Estimated time intervals and rates of viral detection are based on data from several published reports. Because of variability in values among studies, estimated time intervals should be considered approximations and the probability of detection of SARS-CoV-2 infection is presented qualitatively. SARS-CoV-2 indicates severe acute respiratory syndrome coronavirus 2; PCR, polymerase chain reaction. ^{a}Detection only occurs if patients are followed up proactively from the time of exposure. ^{b}More likely to register a negative than a positive result by PCR of a nasopharyngeal swab.

FIG. 6 is an exemplary embodiment of an IgG, IgM, IgA to SARS-CoV-2 proteins fast assay in accordance with the present disclosure.

FIG. 7 is an exemplary embodiment of a patient IgA to SARS-CoV-2 S1 protein fast assay in accordance with the present disclosure.

FIG. 8 is an exemplary embodiment of an IgA epitope map to SARS-CoV-2 S1 in accordance with the present disclosure.

FIG. 9 is an exemplary embodiment of a fast SARS-CoV-2 assay in accordance with the present disclosure.

FIG. 10 is an exemplary embodiment of a patient IgA to SARS-CoV-2 S1-regular assay in accordance with the present disclosure.

FIG. 11 is an exemplary embodiment of a regular SARS-CoV-2 assay in accordance with the present disclosure.

FIG. 12 is an exemplary embodiment of patient IgA to SARS-CoV-2 S1 and RBD fast assay in accordance with the present disclosure.

FIG. 13 is an exemplary embodiment of a fast assay N protein IgG without and with plasmonic-fluor in accordance with the present disclosure.

FIG. 14 is an exemplary embodiment of dilutions of rabbit anti-COVID-19 nucleocapsid antibody in accordance with the present disclosure.

FIG. 15 is an exemplary embodiment of an IgG SARS-CoV-2 protein titers fast assay in accordance with the present disclosure.

FIG. 16A is an exemplary embodiment of a SARS-CoV-2 illustration showing RBD and peptides 553-556 in accordance with the present disclosure. FIG. 16B is an exemplary embodiment of a SARS-CoV-2 illustration showing ACE2 binding region and fusion peptide in accordance with the present disclosure. FIG. 16C is an exemplary embodiment of linear neutralizing epitopes in accordance with the present disclosure.

FIG. 17 is another exemplary embodiment of linear neutralizing epitopes in accordance with the present disclosure.

FIG. 18 is an exemplary embodiment of a multiplexed ELISA for neutralizing antibodies in accordance with the present disclosure.

FIG. 19A is an exemplary embodiment of a multiplexed 96-well format ELISA assay with neutralizing antibody titers in convalescent plasma in accordance with the present disclosure. FIG. 19B is an exemplary embodiment of a multiplexed 96-well format ELISA assay with neutralizing antibody titers in convalescent plasma in accordance with the present disclosure.

FIG. 20 is an exemplary embodiment of neutralizing epitope IgG responses in accordance with the present disclosure.

FIG. 21 is an exemplary embodiment of a SARS-CoV-2 S1 neutralizing antibody to peptide 553-570 in accordance with the present disclosure.

FIG. 22 is an exemplary embodiment of neutralizing epitope total IgA responses in accordance with the present disclosure.

FIG. 23 is an exemplary embodiment of a lateral flow device for assay of patient plasma in accordance with the present disclosure.

FIG. 24 is an exemplary embodiment of a schematic illustration of plasmonic-fluor enhanced epitope-specific SARS-CoV-2 serology assay in a multiplexed manner in accordance with the present disclosure.

FIG. 25A is an exemplary embodiment of a schematic illustration of plasmonic fluor as the ultrabright fluorescence nanolabel comprising of gold nanorod as plasmonic core, polymer spacer layer, fluorophores, and biotin, as the universal recognition element in accordance with the present disclosure. FIG. 25B is an exemplary embodiment of visible-NIR extinction of plasmonic-fluor in accordance with the present disclosure. FIG. 25C is an exemplary embodiment of fluorescence images and corresponding fluorescence intensity of streptavidin-CW800 before and after specific binding of plasmonic-fluor through interaction between biotin and streptavidin, showing a 1500-fold increased fluorescence intensity after applying plasmonic-fluor in accordance with the present disclosure. Data are mean±s.d. a.u., arbitrary units. FIG. 25D is an exemplary embodiment of dose-dependent fluorescence intensity anti SARS-CoV-2 Nucleocapsid (N) protein IgG on microtiter plate by conventional ELISA (black dots) and p-FLISA (red dots) performed in 20 minutes and fluorescence intensity map at various analytes concentration in accordance with the present disclosure. FIG. 25E is an exemplary embodiment of anti SARS-CoV-2 spike protein RBD domain IgG dose-dependent fluorescence intensity on microtiter plate by conventional ELISA (black dots) and p-FLISA (red dots) performed in 20 minutes and fluorescence intensity map at various analytes concentration in accordance with the present disclosure.

FIG. 26A is an exemplary embodiment of dose-dependent fluorescence intensity Anti SARS-CoV-2 spike protein RBD domain IgG on microtiter plate by p-FLISA with the incubation of plasmonic-fluor at OD=0.5 in accordance with the present disclosure. FIG. 26B is an exemplary embodiment of dose-dependent fluorescence intensity Anti SARS-CoV-2 spike protein RBD domain IgG on microtiter plate by p-FLISA with the incubation of plasmonic-fluor at OD=1 in accordance with the present disclosure. FIG. 26C is an exemplary embodiment of dose-dependent fluorescence intensity Anti SARS-CoV-2 spike protein RBD domain IgG on microtiter plate by p-FLISA with the incubation of plasmonic-fluor at OD=1.5 in accordance with the present disclosure. FIG. 26D is an exemplary embodiment of dose-dependent fluorescence intensity Anti SARS-CoV-2 spike protein RBD domain IgG on microtiter plate by p-FLISA with the incubation of plasmonic-fluor at OD=2 in accordance with the present disclosure.

FIG. 27A is an exemplary embodiment of fluorescence intensity obtained with plasmonic-fluor demonstrating IgG response targeting SARS-CoV-2 S protein subunit 1 in individual convalescent patient plasma and healthy control at 1/270 times dilution in accordance with the present disclosure. FIG. 27B is an exemplary embodiment of fluorescence intensity obtained with plasmonic-fluor demonstrating IgG response targeting SARS-CoV-2 N protein in individual convalescent patient plasma and healthy control at 1/270 times dilution in accordance with the present disclosure. FIG. 27C is an exemplary embodiment of fluorescence intensity maps demonstrating the titer of IgG, IgM, IgA response to SARS-CoV-2 S protein subunit 1 (S1), subunit 2 (S2), receptor binding domain (RBD), and Nprotein in convalescent plasma of patient 19, with conventional fluorophores (top) and plasmonic-fluor (bottom) in accordance with the present disclosure.

FIG. 28A is an exemplary embodiment of a graphic titre of IgG targeting SARS-CoV-2 nucleocapsid protein (black) and spike protein subunit 1 (red) in individual COVID-19 convalescent patient 11 plasma as measured by antibody p-FLISA in accordance with the present disclosure. FIG. 28B is an exemplary embodiment of a graphic titre of IgG targeting SARS-CoV-2 nucleocapsid protein (black) and spike protein subunit 1 (red) in individual COVID-19 convalescent patient 12 plasma as measured by antibody p-FLISA in accordance with the present disclosure. FIG. 28C is an exemplary embodiment of a graphic titre of IgG targeting SARS-CoV-2 nucleocapsid protein (black) and spike protein subunit 1 (red) in individual COVID-19 convalescent patient 13 plasma as measured by antibody p-FLISA in accordance with the present disclosure. FIG. 28D is an exemplary embodiment of a graphic titre of IgG targeting SARS-CoV-2 nucleocapsid protein (black) and spike protein subunit 1 (red) in individual COVID-19 convalescent patient 14 plasma as measured by antibody p-FLISA in accordance with the present disclosure. FIG. 28E is an exemplary embodiment of a graphic titre of IgG targeting SARS-CoV-2 nucleocapsid protein (black) and spike protein subunit 1 (red) in individual COVID-19 convalescent patient 15 plasma as measured by antibody p-FLISA in accordance with the present disclosure. FIG. 28F is an exemplary embodiment of a graphic titre of IgG targeting SARS-CoV-2 nucleocapsid protein (black) and spike protein subunit 1 (red) in individual COVID-19 convalescent patient 16 plasma as measured by antibody p-FLISA in accordance with the present disclosure. FIG. 28G is an exemplary embodiment of a graphic titre of IgG targeting SARS-CoV-2 nucleocapsid protein (black) and spike protein subunit 1 (red) in individual COVID-19 convalescent patient 17 plasma as measured by antibody p-FLISA in accordance with the present disclosure. FIG. 28H is an exemplary embodiment of a graphic titre of IgG targeting SARS-CoV-2 nucleocapsid protein (black) and spike protein subunit 1 (red) in individual COVID-19 convalescent patient 18 plasma as measured by antibody p-FLISA in accordance with the present disclosure. FIG. 28I is an exemplary embodiment of a graphic titre of IgG targeting SARS-CoV-2 nucleocapsid protein (black) and spike protein subunit 1 (red) in individual COVID-19 convalescent patient 19 plasma as measured by antibody p-FLISA in accordance with the present disclosure. FIG. 28J is an exemplary embodiment of a graphic titre of IgG targeting SARS-CoV-2 nucleocapsid protein (black) and spike protein subunit 1 (red) in individual COVID-19 convalescent patient 20 plasma as measured by antibody p-FLISA in accordance with the present disclosure. FIG. 28K is an exemplary embodiment of a graphic titre of IgG targeting SARS-CoV-2 nucleocapsid protein (black) and spike protein subunit 1 (red) in healthy control plasma as measured by antibody p-FLISA in accordance with the present disclosure.

FIG. 29A is an exemplary embodiment of a fluorescence intensity maps of IgG targeting SARS-CoV-2 spike protein subunit 1 in individual COVID-19 convalescent patient plasma and healthy control plasma, obtained with application of plasmonic-fluor in accordance with the present disclosure. FIG. 29B is an exemplary embodiment of a fluorescence intensity maps of IgG targeting SARS-CoV-2 nucleocapsid protein in individual COVID-19 convalescent patient plasma and healthy control plasma, obtained with application of plasmonic-fluor in accordance with the present disclosure.

FIG. 30A is an exemplary embodiment of fluorescence intensity maps of IgG targeting SARS-CoV-2 spike protein receptor binding domain in convalescent plasma of patient 13 with conventional fluorophores (left) and plasmonic fluor (right) in accordance with the present disclosure. FIG. 30B is an exemplary embodiment of fluorescence intensity obtained with the application of plasmonic fluor for 90 times (black) and 1440 times (red) dilution of convalescent plasma of patient 13 in accordance with the present disclosure. FIG. 30C is an exemplary embodiment of fluorescence intensity obtained with the application of plasmonic fluor performed with 5 minutes (black) and 60 minutes (red) incubation within each step of the assay in accordance with the present disclosure.

FIG. 31A is an exemplary embodiment of a schematic illustration depicting the structure of peptides encoding the sequence of SARS-CoV-2 neutralizing epitopes and their conjugations with BSA in accordance with the present disclosure. A cysteine was added to allow coupling to the BSA scaffold along with three glycines to project the peptide from the BSA surface. FIG. 31B is an exemplary embodiment of fluorescence intensity and intensity maps obtained before (black) and after (red) application of plasmonic-fluor with various dilution factor of convalescent patient plasma 17 (left) and patient 15 (right), respectively, demonstrating epitope 1 and 2 specific IgG in accordance with the present disclosure. FIG. 31C is an exemplary embodiment of optical density and image of conventional ELISA with various dilution factor of convalescent patient plasma 17 (left) and patient 15 (right), respectively, demonstrating epitope 1 and 2 specific IgG in accordance with the present disclosure. All the assays were accomplished in 20 minutes with 5 minutes incubation for each step.

FIG. 32 is an exemplary embodiment of a schematic illustration depicting the chemical conjugation between BSA and peptide with two types of SARS-CoV-2 specific epitope sequence in accordance with the present disclosure.

FIG. 33A is an exemplary embodiment of fluorescence intensity and an intensity map obtained with application of plasmonic-fluor demonstrating epitope specific IgG response in convalescent patient plasma at the 1/500 dilution in accordance with the present disclosure. The fluorescence intensity of albumin specific antibody in each sample has been subtracted as background from the intensity of epitope-specific antibody appearing in the bar graphs. FIG. 33B is an exemplary embodiment of fluorescence intensity and an intensity map obtained with application of plasmonic-fluor demonstrating epitope specific IgA response in convalescent patient plasma at the 1/500 dilution in accordance with the present disclosure. The fluorescence intensity of albumin specific antibody in each sample has been subtracted as background from the intensity of epitope-specific antibody appearing in the bar graphs. FIG. 33C is an exemplary embodiment of fluorescence intensity maps of IgA (top row) and IgA1 (bottom row) targeting BSA and BSA-peptide (epitope 1) with 1/500 dilution of convalescent patient plasma in accordance with the present disclosure.

FIG. 34A is an exemplary embodiment of a scheme illustrating arrangement of spatially multiplexed detection of epitope-specific antibodies in accordance with the present disclosure. FIG. 34B is an exemplary embodiment of a Log₁₀ titer of IgG in convalescent patient plasma targeting epitope 1 (black) and epitope 2 (red), measured by antibody p-FLISA in accordance with the present disclosure. FIG. 34C is an exemplary embodiment of fluorescence intensity mapping after application of plasmonic-fluor showing the spatial multiplexed detection of IgG targeting different epitopes in individual convalescent patient plasma and healthy control in accordance with the present disclosure.

FIG. 35A is an exemplary embodiment of a graphic titre of IgG targeting SARS-CoV-2 specific epitope 1 (black) and epitope 2 (red) in individual COVID-19 convalescent patient 11 plasma, testing with spatially multiplexed antibody p-FLISA in accordance with the present disclosure. FIG. 35B is an exemplary embodiment of a graphic titre of IgG targeting SARS-CoV-2 specific epitope 1 (black) and epitope 2 (red) in individual COVID-19 convalescent patient 12 plasma, testing with spatially multiplexed antibody p-FLISA in accordance with the present disclosure. FIG. 35C is an exemplary embodiment of a graphic titre of IgG targeting SARS-CoV-2 specific epitope 1 (black) and epitope 2 (red) in individual COVID-19 convalescent patient 13 plasma, testing with spatially multiplexed antibody p-FLISA in accordance with the present disclosure. FIG. 35D is an exemplary embodiment of a graphic titre of IgG targeting SARS-CoV-2 specific epitope 1 (black) and epitope 2 (red) in individual COVID-19 convalescent patient 14 plasma, testing with spatially multiplexed antibody p-FLISA in accordance with the present disclosure. FIG. 35E is an exemplary embodiment of a graphic titre of IgG targeting SARS-CoV-2 specific epitope 1 (black) and epitope 2 (red) in individual COVID-19 convalescent patient 15 plasma, testing with spatially multiplexed antibody p-FLISA in accordance with the present disclosure. FIG. 35F is an exemplary embodiment of a graphic titre of IgG targeting SARS-CoV-2 specific epitope 1 (black) and epitope 2 (red) in individual COVID-19 convalescent patient 16 plasma, testing with spatially multiplexed antibody p-FLISA in accordance with the present disclosure. FIG. 35G is an exemplary embodiment of a graphic titre of IgG targeting SARS-CoV-2 specific epitope 1 (black) and epitope 2 (red) in individual COVID-19 convalescent patient 17 plasma, testing with spatially multiplexed antibody p-FLISA in accordance with the present disclosure. FIG. 35H is an exemplary embodiment of a graphic titre of IgG targeting SARS-CoV-2 specific epitope 1 (black) and epitope 2 (red) in individual COVID-19 convalescent patient 18 plasma, testing with spatially multiplexed antibody p-FLISA in accordance with the present disclosure. FIG. 35I is an exemplary embodiment of a graphic titre of IgG targeting SARS-CoV-2 specific epitope 1 (black) and epitope 2 (red) in healthy control plasma, testing with spatially multiplexed antibody p-FLISA in accordance with the present disclosure.

FIG. 36A is an exemplary embodiment of a schematic illustration depicting the experiment measuring the enhancement factor of plasmonic-fluor compared to conventional fluorophores and the background signal generated by conventional fluorophores and plasmonic-fluor in accordance with the present disclosure. FIG. 36B is an exemplary embodiment of fluorescence intensity of conventional fluorophore and plasmonic-fluor with OD 0.5, 1, 1.5, and 2, applied to BSA and BSA coated wells in accordance with the present disclosure.

### DETAILED DESCRIPTION OF THE DISCLOSURE

Severe acute respiratory syndrome coronavirus-2 (SARS-CoV-2) has rapidly spread and resulted in global pandemic of COVID-19. Although IgM/IgG serology assay has been widely used, with the entire spike or nucleocapsid antigens, they only provide coarse information about infection stage and future immune protection. Novel technologies enabling easy-to-use and sensitive detection of multiple specific antibodies simultaneously will facilitate precise diagnosis of infection stages, prediction of clinical outcomes, and evaluation of future immune protection upon vial exposure or vaccination. Herein, a rapid and ultrasensitive quantification method is demonstrated for epitope-specific antibodies, including different isotypes and subclasses, in a multiplexed manner. Using an ultrabright fluorescent nanolabel, plasmonic-fluor, this novel assay can be completed in 20 minutes and, more importantly, the LOD of the plasmon-enhanced immunoassay for SARS-CoV-2 antibodies is up to 100-fold lower compared to the assays relying on enzymatic amplification of colorimetric signal. Using convalescent patient plasma, this biodetection method demonstrates the patient-to-patient variability in immune response as evidenced by the variations in whole protein and epitope-specific antibodies. This cost-effective, rapid and ultrasensitive plasmonically-enhanced multiplexed epitope-specific serology is enabled for broad employment to advance epidemiology studies, improve clinical outcomes, and predict future protection against the SARS-CoV-2 prototype and its variants.

### Rapid Plasmonically-Enhanced Detection of Corona Virus Disease (COVID-19)

Existing HRP-linked colorimetric assays for ELISA of immunoglobulins take hours (typically 2-4 hours) but if rushed to 20 minutes are relatively insensitive (Limit of Detection about 1.5 ng/ml IgG). The plasmonic-fluor assays described herein provide at least 25-fold more sensitivity (Limit of Detection 0.06 ng/ml IgG) in a span of 20 minutes. A 2-hour plasmonic-fluor enhanced assay further lowers the limit-of-detection. This is advantageous as less abundant IgA (compared to IgG) immunoglobulins may be more important to patient treatment with convalescent plasmas. Also advantageous is the ability to detect IgG and IgM antibodies to nucleocapsid protein early on after infection. As described herein, IgG, IgM and IgA immunoglobulins are measured that recognize SARS-CoV-2 nucleocapsid, Spike protein S1, Spike protein S2 and the Receptor Binding Domain (RBD) of Spike protein S1.

A comparison of fast (20 minute) and regular (3-4 hour) and classical HRP-colorimetric assay for SARS-CoV-2 nucleocapsid protein IgG ELISA demonstrates superior sensitivity when plasmonic-fluor is present. Additionally, plasmonic-fluor assay of convalescent patient plasma indicates at least 100-fold enhancement of assay sensitivity for IgG, IgM and IgA reaction to SARS-CoV-2 proteins. Applicability of the biosensors, methods, and assays described herein includes rapid identification of individuals with antibodies to SARS-VoV-2 proteins following infection, population screening to determine exposure regardless of symptomology, and phenotyping immunoglobulins of convalescent plasma for optimum patient treatment. In some embodiments, either the 20 minute or 2-hour plasmonic-fluor assay are adaptable to high throughput for true population screening for immune response.

Background COVID-19, an infectious disease caused by severe acute respiratory syndrome coronavirus-2 (SARS-CoV-2), has become a global public health challenge. Most of the current accurate diagnostic techniques (e.g., RT-PCR) are relatively expensive, require equipment that are incompatible with remote and resource-limited regions where disease surveillance and control are critically needed. Moreover, these methods are limited to diagnosis of the disease over a short time window as the viral load becomes undetectable within 2-3 weeks after the onset of infection. Thus, there is a dire need for a rapid, sensitive, and accurate biosensor for COVID-19 that can be broadly deployed to rapidly assess the epidemiology of the disease and limit its outbreak. Design and demonstration of a rapid and sensitive test can greatly facilitate our understanding of (i) the appearance and evolution of the immune response of a person and/or a population infected with SARS-CoV-2; and (ii) epidemiology of this infectious disease, which can possibly identify methods to limit the widespread transmission of the infection.

Highly sensitive and specific serological tests are extremely important for rapidly monitoring the presence and relative concentrations of target antigen-specific antibodies in serum. It is known that the antibody repertoire in the serum represents the record of exogenous agents such as pathogens and vaccines among other factors. Highly sensitive and specific serological tests provide better understanding of the extent of transmission and epidemiology of the infectious disease compared to methods involving the quantification of viral load (e.g., detection of viral RNA copy numbers using RT-PCR), which is known to be undetectable within a few weeks after the infection. Specifically, in the case of SARS-CoV-2, the viral load was found to become undetectable in within 14 days after onset of symptoms. While serologic tests are attractive for both detection and, more importantly, epidemiological studies, the existing biosensors based on enzyme-linked immunosorbent assays (ELISAs) and lateral flow assays (LFAs) are not sufficiently sensitive to identify mild and asymptomatic infections. These considerations suggest the urgent need for more sensitive and rapid serologic tests that can minimize false negatives in detecting and quantifying antibody levels for high-throughput surveillance of COVID-19.

To overcome the limited sensitivity, a novel biosensor (i.e., a newly developed ultrabright fluorescent nanostructure) based on plasmonic-fluor was designed and demonstrated. The novel biolabel enables 100-fold enhancement in the detection sensitivity of SARS-CoV-2 antibody compared to conventional ELISA. Furthermore, the test is easily adapted in POC and resource-limited settings owing to the significantly improved signal-to-noise ratio, which significantly lowers the complexity of the read-out instrumentation.

Design and realization of a highly sensitive and specific serological test for the detection of host IgM/IgG to SARS-CoV-2 using plasmonic-fluors as biolabels are disclosed herein. More specifically, design and demonstration of ultrasensitive detection of SARS-CoV-2 IgG and IgM in spiked human serum samples using plasmonic-fluor as an ultrabright signal reporter, as well as realization of a multimodal ultrasensitive lateral flow assay broadly deployed to understand the transmission of the SARS-CoV-2 across broad populations are disclosed herein.

The present disclosure demonstrates and establishes several novel principles in serological biosensors for the first time. This is the first use of an ultrabright plasmonically-enhanced fluorescent nanoconstruct in detecting extremely low concentrations of target antibodies in biofluids such as human serum. The use of plasmonic-fluor in standard microtiter platform lowers the limit-of-detection by more than two orders of magnitude compared to the conventional serological assays such as ELISAs, enabling more accurate detection and quantification of the target antibodies. As described herein, a dual-modal lateral flow assay is demonstrated for the first time that enables simultaneous detection of relatively high concentrations of the target analyte as a simple colorimetric signal and low concentrations of the analytes through highly sensitive fluorescence signal. The dual-modal lateral flow assay seeks to combine the advantages of simplicity and high sensitivity into a single assay to achieve low false negatives while still operating in point-of-care and resource-limited settings. The biosensing principles established herein are broadly applicable for the detection of a wide range bioanalytes including protein biomarkers, nucleic acids and metabolites. Disclosed is a novel technique to rapidly assess the immune response to the novel virus across a large population in a high-throughput manner and understand the epidemiology of this highly contagious disease.

Results and Discussion

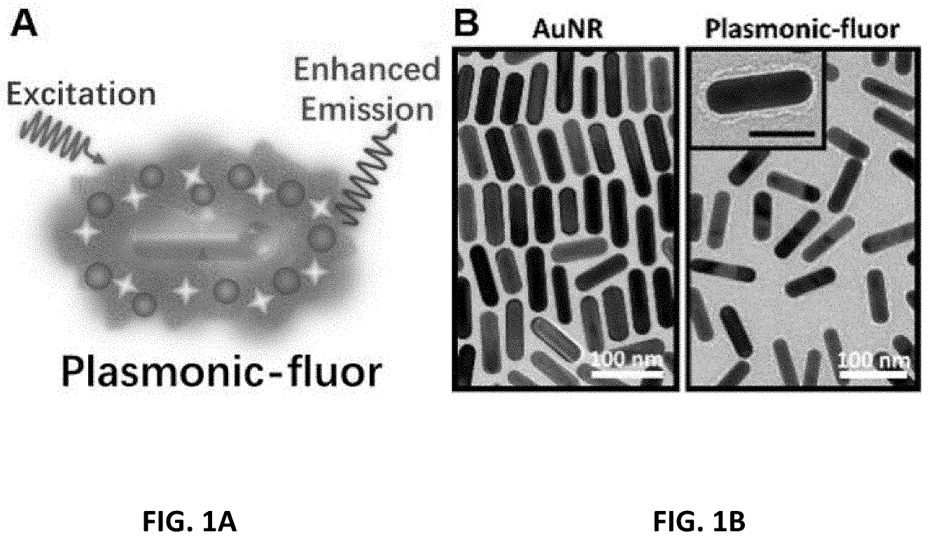

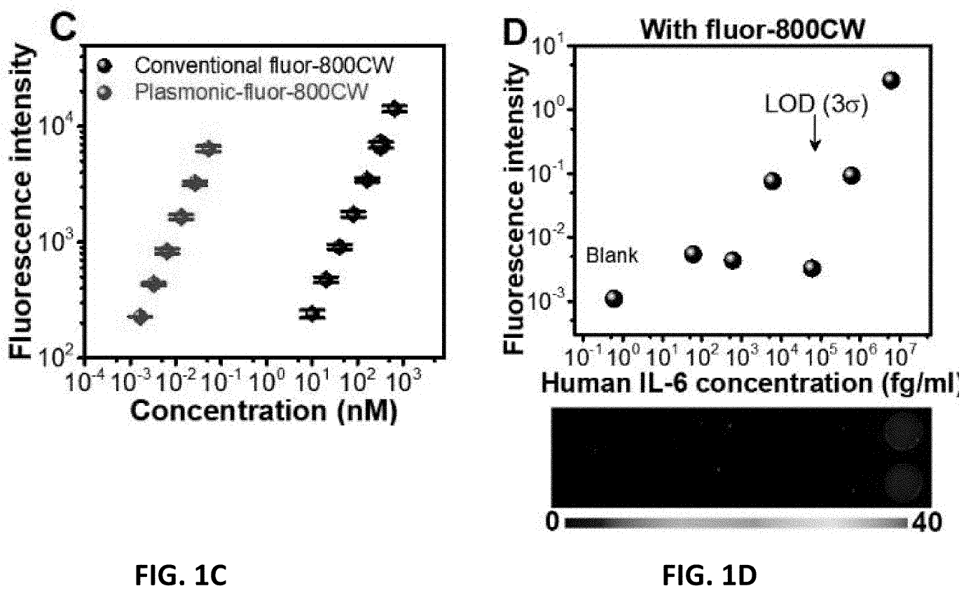

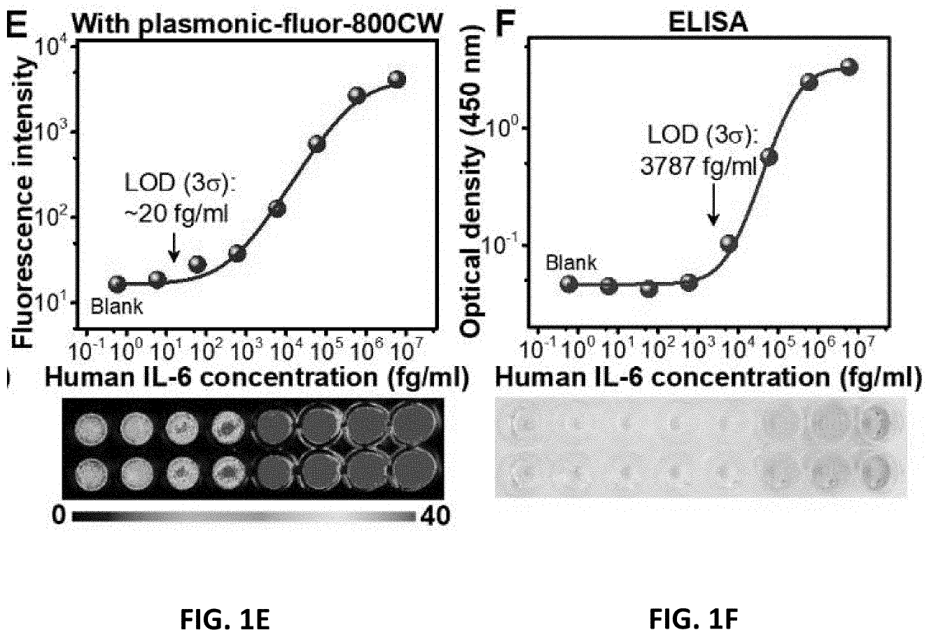

Plasmonic-fluor as an ultrabright nanolabel. To surmount the challenges associated with limited brightness of existing fluorescent biolabels, a novel plasmonic nanoconstruct has been developed, termed plasmonic-fluor. Plasmonic-fluor is comprised of a plasmonic nanostructure as fluorescence enhancer (e.g. gold nanorod (AuNR)), a light emitter (e.g., molecular fluorophores, quantum dots), polymeric spacer layer, and a universal biological recognition element (e.g. biotin) (FIG. 1A, B). The plasmonic-fluor exhibited up to 6700 (±900)-fold brighter signal compared to the corresponding single near infrared fluorophore (800CW) and vastly outperforms existing nanoengineered fluorescent structures (FIG. 1C).

Plasmonic-fluor enhanced ultrasensitive fluoroimmunoassay. Efficacy of plasmonic-fluor has been tested as a nanolabel for realizing an ultrasensitive plasmon-enhanced fluorophore-linked immunosorbent assay (p-FLISA) implemented on a standard microtiter plate. Human interleukin 6 (IL-6), a pro-inflammatory cytokine, was employed as a representative protein biomarker. Conventional FLISA involves a standard sandwich format of capture antibody, analyte (IL-6), biotinylated detection antibody, followed by exposure to streptavidin-fluorophore (e.g., 800CW). In p-FLISA, plasmonic-fluor-800CW is introduced to bind with streptavidin as the signal enhancer. Fluorescence signal obtained after applying the plasmonic-fluor-800CW revealed nearly 1440-fold enhancement in the ensemble fluorescence intensity compared to the conventional FLISA at the highest analyte concentration tested here (6 ng/ml) (FIG. 1D, E). The limit-of-detection (LOD, defined as mean+3σ of the blank) of conventional FLISA was calculated to be ˜95 pg/ml (FIG. 1D). Surprisingly however, fluorescence signal with p-FLISA was detectable down to 20 fg/ml of IL-6 (˜1 fM) (FIG. 1E), which represents a 4750-fold improvement in the LOD compared to conventional FLISA. The LOD and lower limit of quantification ((LLOQ), defined as mean+10σ of the blank, ˜82 fg/ml) of p-FLISA were found to be 189-fold and 120-fold lower than the “gold standard” enzyme-linked immunosorbent assay (ELISA), which involves enzymatic amplification of the colorimetric signal (FIG. 1F). In addition, p-FLISA exhibited a dynamic range (ratio between higher and LLOQ) of five orders of magnitude, which is more than two-orders of magnitude higher than that of ELISA.

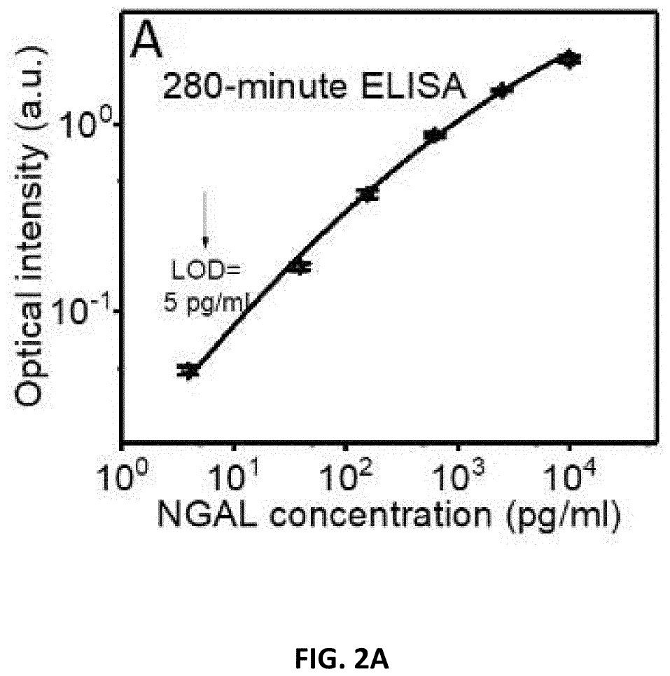

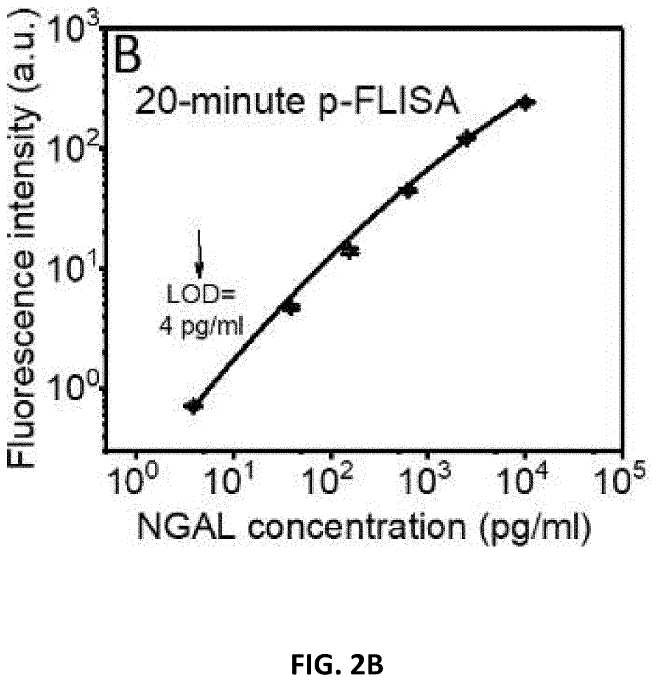

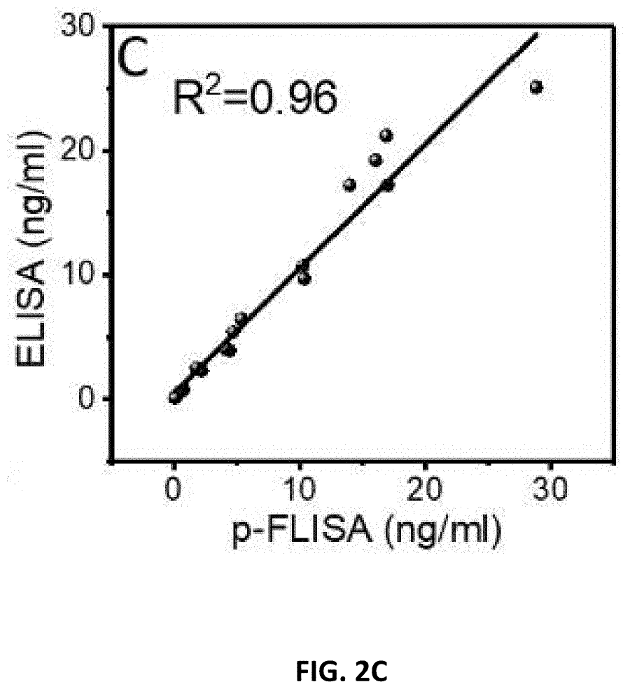

Plasmonic-fluor enhanced ultrafast fluoroimmunoassay. The p-FLISA is implementable in a much shorter time compared to ELISA. Concentrations of urinary neutrophil gelatinase-associated lipocalin (NGAL), a biomarker for AKI, was measured in nine kidney disease patients as well as nine healthy individuals using a 20-minute p-FLISA. In the 20-minute p-FLISA, the reagent incubation time during each step (sample, detection antibody, streptavidin, and plasmonic-fluor) was significantly shortened to 5 minutes. Notably, the 20-minute ultrafast p-FLISA achieved the same sensitivity as the conventional ELISA, which requires a total of 280-minute incubation of these reagents (FIG. 2A, B). This ultrafast p-FLISA was able to quantitatively measure urinary NGAL concentrations from all healthy individuals and kidney disease patients and the assay revealed that NGAL concentrations in kidney disease patients to be significantly higher (by more than 10-fold) compared to that of the healthy individuals (data not shown). Moreover, the NGAL concentrations determined using 20-minute p-FLISA showed a linear correlation (R2=0.96) with those acquired from the standard 280-minute ELISA, proving that the accuracy of the ultrafast assay is not compromised (FIG. 2C). ELISA, however, showed significantly deteriorated performance (LOD: 625 pg/ml) when the overall assay time was shortened to 20 minutes, and failed to detect NGAL concentrations in a few of the patient samples as well as all of the healthy volunteers.

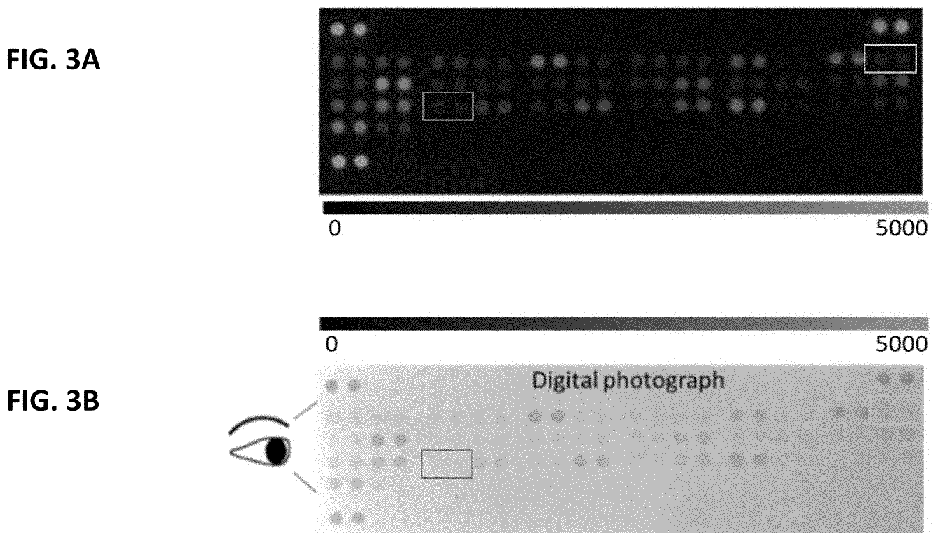

Dual-modal ultrasensitive detection using plasmonic-fluor. In addition to ultrabright fluorescence signal, plasmonic-fluor acts as a visible nanolabel. Proteomic array comprised of antibodies to biomarkers of human kidney disease was employed as a representative example. Human urine sample from a patient with kidney disease was mixed with biotinylated detection antibody cocktail and added onto the nitrocellulose membrane of the array. The membrane was exposed to streptavidin and plasmonic-fluor. The plasmonic nanostructures exhibit large extinction cross-section, which can be up to 5-6 orders of magnitude larger than light absorption of most organic dyes. This unique property renders the possibility of utilizing plasmonic-fluors as multimodal bio-label. Indeed, the binding of plasmonic-fluor to the sensing domains resulted in analyte concentration-dependent color spots, which are directly visualized by the naked eye (FIG. 3B). The color intensity showed good correlation with its corresponding fluorescence intensity (FIG. 3A). However, for low abundant protein marker, colorimetric signals are extremely weak or even become non-detectable (FIG. 3A) compared to their corresponding fluorescence signals (FIG. 3B) (highlighted using colored boxes), indicating that dual-modal sensing is required to enable simultaneous detection of relatively high concentrations of the target analyte as a simple colorimetric signal and low concentrations of the analytes through highly sensitive fluorescence signal.

Methods

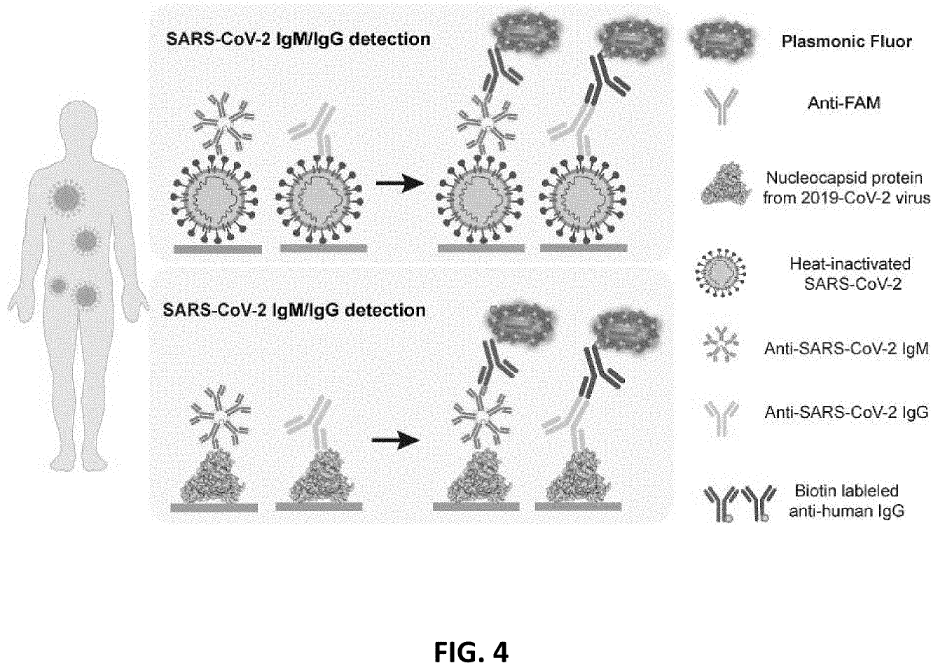

Design and demonstrate ultrasensitive detection of SARS-CoV-2 IgG and IgM using plasmonic-fluor as a signal reporter. Following the SARS-CoV-2 infection, ensuing host IgM/IgG response to SARS-CoV-2 (which evolves within one to two weeks) can serve as the basis of diagnostic assays and epidemiological studies of COVID-19, providing a longer detection window. Heat-inactivated SARS-CoV-2 was utilized as a recognition element for specific capture of the target IgG and IgM (FIG. 4). The heat-inactivated SARS-CoV-2 immobilized at the bottom of standard microtiter plates followed by exposure to the sample containing IgG and IgM generated by the host, which results in specific capture of the IgG and IgM. Subsequently, the microtiter plates were exposed to biotinylated anti-human IgG or anti-human IgM, streptavidin, and plasmonic-fluors in a sequential manner. The fluorescence intensity was measured using commonly available fluorescence imaging system (e.g. LI-COR CLx imager).

The following bioanalytical parameters of the plasmonically-enhanced IgG/IgM fluoroimmunoassay were evaluated: 1) Sensitivity: To determine the assay sensitivity and limit-of-detection (LOD), defined as the analyte concentration corresponding to the mean fluorescence intensity of blank plus three times of its standard deviation, serial dilutions of IgG or IgM with known concentrations as well as blank control (1% BSA) were employed as standards to obtain the dose-responsive curve. Specific antibodies (e.g., from Creative Diagnostics (Anti-SARS-CoV-2 N protein monoclonal antibody (CABT-RM320))) were diluted using 1% BSA. Simultaneously, gold standard ELISA of target antibody was performed, which involves streptavidin conjugated with horseradish peroxidase (HRP), and its sensitivity and limit-of-detection compared with plasmonically-enhanced fluoroimmunoassay. In addition to the measurement of absolute concentrations, the antibody titer was determined, defined as the inverse of the highest serum dilution that yields a signal above a chosen cut-off value (e.g., twice the mean value of the blank wells), using plasmon-enhanced fluoroimmunoassay and ELISA. The sensitivity of the two assays was also compared based on the measured titer number. Considering the ultra-brightness and specificity of plasmonic-fluor, plasmon-enhanced fluoroimmunoassay offers significantly higher sensitivity and lower limit-of-detection than ELISA. 2) Turnaround time: the minimal assay time was determined without compromising the assay sensitivity by shortening the incubation time of both target antibody and biotinylated anti-human IgG. 3) Dynamic range: The immunoassay has a lower and upper detection limit that signifies the range in which the fluorescence intensity changes monotonically with the change in analyte concentration. It is important that the working range of fluoroimmunoassay includes a wide concentration range of the target antibody to account for large variations that exist across populations (individual-to-individual variations) and different times with respect to onset of infection for the same individual. A broad range of antibody concentrations was tested to measure the dynamic range of plasmonically-enhanced fluoroimmunoassay. 4) Reliability: As validation of the plasmon-enhanced fluoroimmunoassay, blind tests were performed using healthy serum spiked with antibody. Specifically, a range of known concentrations of target antibody was prepared, and subsequently diluted by 10-fold using 1% BSA solution and measured using both plasmonically-enhanced fluoroimmunoassay and ELISA. The correlations between measured concentrations of the antibody and known spiked concentrations were obtained and compared with that using ELISA. 5) Reproducibility: To ensure scientific rigor, each test was repeated at least 5 times to determine the standard deviation of the test results. The measurement of bioanalytical parameters was performed at least three times independently (on different days with different batches of plasmonic-fluor) to determine the assay reproducibility. An alternative approach was to employ nucleocapsid protein from SARS-CoV-2 virus (RayBiotech, catalog #230-01104) as biorecognition element for specific capture of the target IgG and IgM (FIG. 4). Various bioanalytical parameters, such as sensitivity, dynamic range, reliability, and reproducibility were further evaluated using methods to those similar described herein above.

Design and demonstrate “dual-modal” ultrasensitive lateral flow assay for rapid detection of SARS-CoV-2 IgG and IgM. Lateral flow assay (LFA) provides a simplified method to detect the presence of SARS-CoV-2 IgG and IgM in biofluids of interest. However, there is an urgent need for enhancing the sensitivity of LFAs to detect the presence of these biomarkers at earlier stage of disease progression and at a longer detection window to reduce false negative rate. A key limiting factor in assay sensitivity is the method of readout. Commonly, LFAs display relies on colorimetric signal from gold nanostructures, owing to their large absorption and scattering cross sections. However, visual readout is often unreliable at the lower threshold of analyte concentrations and therefore provides limited potential to effectively identify SARS-CoV-2 IgG and IgM at an early stage. As described herein, plasmonic-fluor was employed as the signal reporter to significantly improve the sensitivity of IgM/IgG lateral flow assay. Notably, plasmonic-fluor exhibits both fluorescence and colorimetric signals as discussed herein. Commercially available LFA assembly kits (Claremont Bio, Product code #07.600.01) with necessary components (e.g. nitrocellulose, sample pads, absorbent pads) were acquired, and specifically, nucleocapsid protein from SARS-CoV-2 virus (RayBiotech, catalog #230-01104) was employed as the biorecognition element and immobilized on the nitrocellulose membrane in LFA. As comparison, conventional LFA involving pure gold nanoparticles as the signal reporter was performed simultaneously. Sensitivities of the two lateral flow assays were tested and compared using target antibody (Anti-SARS-CoV-2 N protein monoclonal antibody from Creative Diagnostics (CABT-RM320)) with serial dilutions. Optical intensity of LFAs were recorded by digital camera, and fluorescence intensity were recorded using common fluorescence imager (e.g. LI-COR CLx). Considering the ultrahigh fluorescent signal and large absorption/scattering cross sections of plasmonic-fluor, it enables significantly lower limit-of-detection of LFA based on fluorescence readout mode without compromising the colorimetric signal at high antibody concentrations.

Outcomes. Outcomes included (i) a comprehensive understanding of the efficiency of plasmonic-fluor in enhancing the bioanalytical parameters (e.g., limit-of detection, dynamic range, sensitivity); (ii) a successfully designed and realized novel biosensor for highly sensitive detection of SARS-CoV-2 IgG and IgM compared to conventional bioassays such as ELISA; and (iii) a dual-modal lateral flow assay for the detection of high concentrations of SARS-CoV-2 IgG and IgM using a colorimetric signal and low concentrations of SARS-CoV-2 IgG and IgM using fluorescence signal from the plasmonic-fluors. The disclosed novel sensing platform is easily adapted to a broad range of public health threats, allowing for rapid development of ultrasensitive and low-cost biosensing technologies in resource-limited regions.

Development of an Ultra-Sensitive, Fast, Plasmon-Enhanced Assay for SARS-CoV-2.

Specific Immunoglobulins. Severe acute respiratory syndrome coronavirus 2 (SARS-CoV-2), also known as COVID-19, is a readily transmitted disease that has become a global health threat. Accurate diagnosis of SARS-CoV-2 infection, or any emerging threat, requires an adaptable assay platform that can be readily customized to detect signature threat biomolecules/biomarkers. The problem is that current SARS-CoV-2 diagnostics, and more generally many disease diagnostics, are usually time-consuming, expensive, and require exquisite fine-tuning customization to develop. Even though lateral flow devices are rapid, they are usually insensitive and typically have a problem with specificity apart from their time to develop. This presents several challenges, including i) time for assay development, ii) time for assay validation, iii) assay costs, and iv) analytical throughput. Additionally, SARS-CoV-2 and other members of the Corona virus family have similar symptomology, thrive in similar geography, and are readily transmittable, making precise diagnostics and diagnoses difficult.

As described herein, a highly innovative, rapidly deployable assay platform was created to be swiftly adaptable to the diagnosis of any disease, and specifically including SARS-CoV-2. The platform is based on suitably functionalized gold nanostructures that serve as nano-antenna to amplify light emitted from a standard fluorophore used in traditional fluorescent-based sandwich immunoassays. Further described herein is the identification of immune epitopes in the Spike protein 1 (S1) and its receptor binding domain (RBD) specific to SARS-CoV-2 and differentiation of these from immune epitopes common to the corona virus family. Following development of the analytical platform and the ability to select signature immune epitopes of SARS-CoV-2 infection, molecular diagnostic assays were developed to efficiently, rapidly, cost-effectively and reliably diagnose SARS-CoV-2 infection in sera. These were based on the immunologic responses (IgG, IgM and IgA) of individuals exposed to the virus regardless of their symptoms.

Develop and validate a rapid, ultra-sensitive plasmonic-fluor-based SARS-CoV-2-related IgM/IgG/IgA diagnostic assays. Suitably functionalized gold nanostructures provide an innovative, highly modifiable diagnostic platform for tuning assay sensitivity and specificity to that required to detect selected signature biomarkers of disease (i.e., IgG/IgM/IgAt), which can be rapidly customized in response to emerging biothreats, herein SARS-CoV-2.

Methods. Assay conditions were optimized for the ability of plasmonic-fluor nanotransducers for infectious disease immune analytes (IgG, IgM and IgA), to improve analytical speed, sensitivity and specificity of SARS-CoV-2 assays. This approach was grounded on the ability to create a sensitive and specific assay for IgG/IgM during Zika infection. ELISAs functionalized with whole SARS-Co-V-2 S1 protein and the S1 RBD as “bait” were used to capture the immune globulins that were individually measured with specific biotinylated anti-human IgG, IgM or IgA detectors and the fluorescent signal amplified by plasmonic-fluor. Existing ELISA assays for SARS-CoV-2 infection utilize similar bait protein as capture reagent. However, due to the similarity of these proteins with other related corona viruses such as SARS-CoV-1, a plaque reduction neutralization test or a PCR assay is usually required to conclusively distinguish SARS-CoV-2 infection from that due to other viruses of the family. The assay described herein shortens the time and reduces the cost to gain an accurate diagnosis and is usable with other assays.

Develop and validate specific biorecognition capability for SARS-CoV-2 immune recognition by epitope mapping of SARS-CoV-2. Identification of IgG, IgM and IgA immune epitopes specific to SARS-CoV-2 adds a specificity to conventional ELISA assays that is lacking in existing commercially available assays.

Methods: Commercially available S1 epitope arrays were used to screen convalescent plasmas and identify epitopes recognized by the IgG, IgM and IgA present in the plasma using plasmonic-fluor to boost assay sensitivity. Based on this screen using limited and non-overlapping peptides, commercially available epitope arrays comprised of overlapping peptides of all SARS-CoV-2 proteins were utilized to identify signature peptides in the SARS-CoV-2 S1, S2, nucleocapsid (N) protein and the RBD of S1. Numerous existing assays measure the IgG/IgM response of N protein, the least diverse protein of the corona virus family, thus leading to a lack of specificity. Finding an epitope or two unique to SARS-CoV-2 N protein boosted assay sensitivity significantly, especially that of lateral flow devices while providing the requisite specificity.

Consequently, a public health issue was addressed and an unmet need was fulfilled to develop cost-efficient assays for specific disease screening, early diagnosis, and rapid treatment, particularly for the use of convalescent plasmas. Additionally, poor specificity of traditional target capture strategies was overcome. This was transformative by eliminating the unintended background of antibodies common to most if not all corona viruses due to protein sequence similarities. The nanotechnology-based assay described herein for SARS-CoV-2 infection is deployable in resource limited settings to a hand-held smart phone interface thus extending the global reach of this technology.

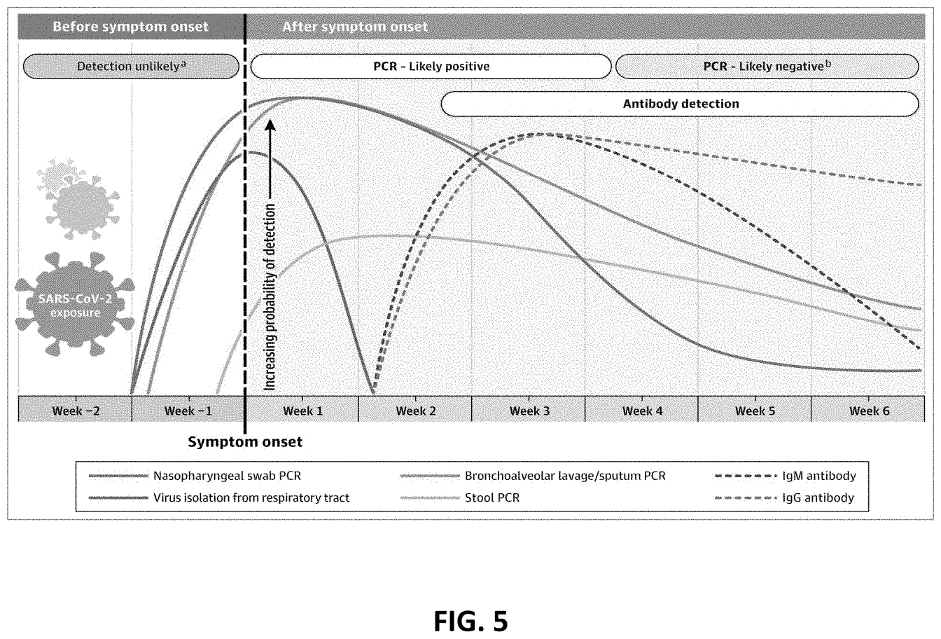

SARS-CoV-2 Infection. SARS-COV-2, a single-stranded RNA member of the Corona virus family, is primarily transmitted between humans by aerosolized droplets through the air and by direct touch or touch of infected surfaces. SARS-COV-2 is initially translated as a single polyprotein which is then cleaved post-translationally. Major antigenic markers for SARS-CoV-2 infection are Spike protein 1 (S1) Spike protein 2 (S2) and the nucleocapsid protein (N) which elicit an ensuing host IgM/IgG/IgA response which serves as a basis of diagnostic assays for SARS-COV-2 infection. The general timeline for the appearance of corona virus events is depicted in FIG. 5. Thus, detection of the IgM, IgA and IgG response to SARS-COV-2 affords diagnosis within a week and beyond of infection.

Typically, assays for the immune response measure IgM and IgG. However, immunoglobulin A (IgA) has a very important role in the adaptive humoral immune protection at mucosal surfaces, particularly in the respiratory and gastrointestinal systems. Secretory IgA is primarily dimeric linked by a short joining chain and a longer secretory chain. On mucosal surfaces, IgA neutralizes bacterial and viral attempts at infection. However, serum IgA is monomeric and is the second most abundant immunoglobulin in the sera after IgG with both pro-inflammatory and anti-inflammatory properties. Human IgA has two forms; IgA1 and IgA2 that differ in the hinge region and the number of glycosylation sites. In the sera, IgA1 predominates but in mucosal secretions, both isotypes have similar concentrations. Following infection and antigen presentation to T helper cells, B cells maturate by class switch recombination to change from producing IgM to produce IgA or IgG. As described herein, measurements included IgG, IgM and IgA, the latter due to its relevance to the respiratory system. Also, typically, the IgG and IgM assays lack data to support their clinical utility due to insufficient sensitivity and specificity.

Therefore, a highly innovative, easily deployable nanoparticle-based platform technology is described herein, built on suitably functionalized gold nanostructures, swiftly adaptable to molecular diagnosis of any disease termed plasmonic-fluor in both a patch and soluble form. The nanoparticle itself is in essence a ‘Swiss Army Knife’ with interchangeable core fluorophores that are tunable to amplify fluorescence of any commercially available assay (e.g., FITC, Cy3, Cy5, 680LT, 800CW) to meet the label-free specificity and sensitivity required to measure any disease marker of interest. The nano-assays proposed herein utilized the soluble form of plasmonic-fluor and are the first application and proof-of-concept of this technology for rapid creation of an infectious disease diagnostic.

Develop and validate a rapid, ultra-sensitive plasmonic-fluor-based SARS-CoV-2-related IgM/IgG/IgA diagnostic assays. As described herein, suitably functionalized gold nanostructures provide an innovative, highly modifiable, label-free diagnostic platform for adjusting assay sensitivity to that required to detect SARS-COV-2 infection by means of detecting IgG, IgM and IgA directed to SARS-CoV-2 proteins such as S1, S2 or N. This technology is rapidly customized in response to emerging bio-threats as needed.

Methods. Size and shape of the plasmonic nanotransducers was optimized for a given analyte and biorecognition element, to improve the analytical and clinical sensitivity to measure the analyte. This approach was grounded on the ability to create LSPR-based assays for biomarkers of non-infectious disease, and in the ability to measure the IgG/IgM response to patients infected with Zika virus. Studies have measured an IgA response in SARS-CoV-2 infected patients to S1 and/or RBD.

Plasmonic Nanotransducers. Plasmonics involve control of light at nanoscale by using surface plasmons. Design and synthesis of various size- and shape-controlled nanostructures maximizes the sensitivity and to quantitatively measure each analyte in the relevant biologic concentration range. Additionally, designing and optimizing biofunctionalization strategies and choice of “bait” to operate within the optimum electromagnetic field of nanoparticles augments assay sensitivity. Thus, the sensitivity of a SARS-COV-2 S1 or RBD based assay is tunable to optimize diagnostic sensitivity while preserving diagnostic specificity. In this case, analyte (IgG, IgM or IgA) concentrations are readily quantified by the fluorescence as a function of plasma dilution or immunoglobulin titter. Plasmonic-fluors were prepared to amplify signals from commercially available streptavidin-linked 800CW and 680 LT (LI-COR). Steps to authenticate assembly of the plasmonic-fluor were monitored by absorbance spectrophotometry, transmission (TEM) and scanning electron microscopy (SEM) and atomic force microscopy (AFM).

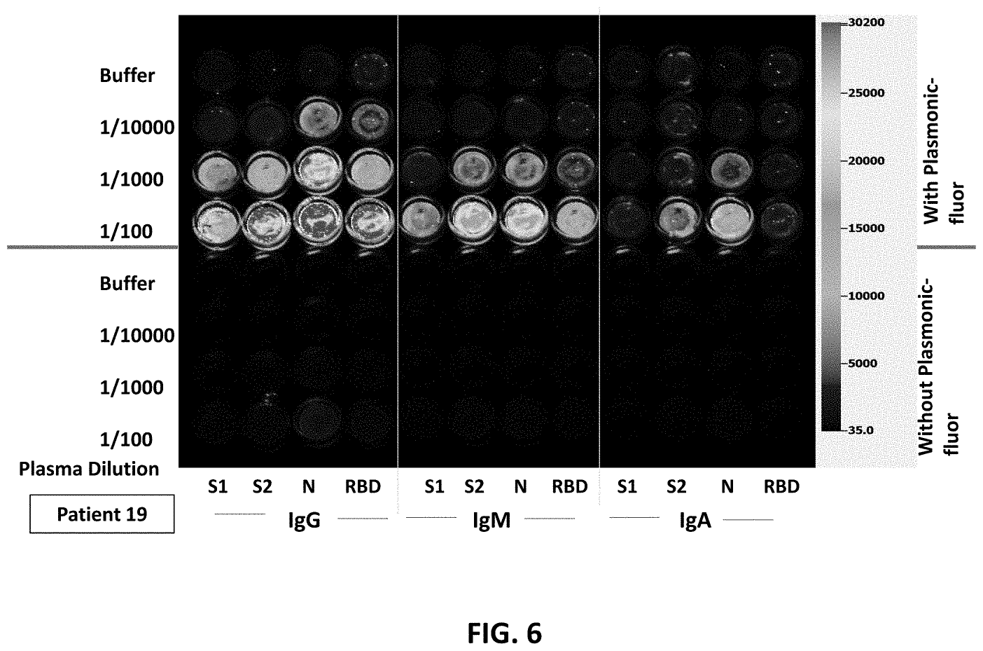

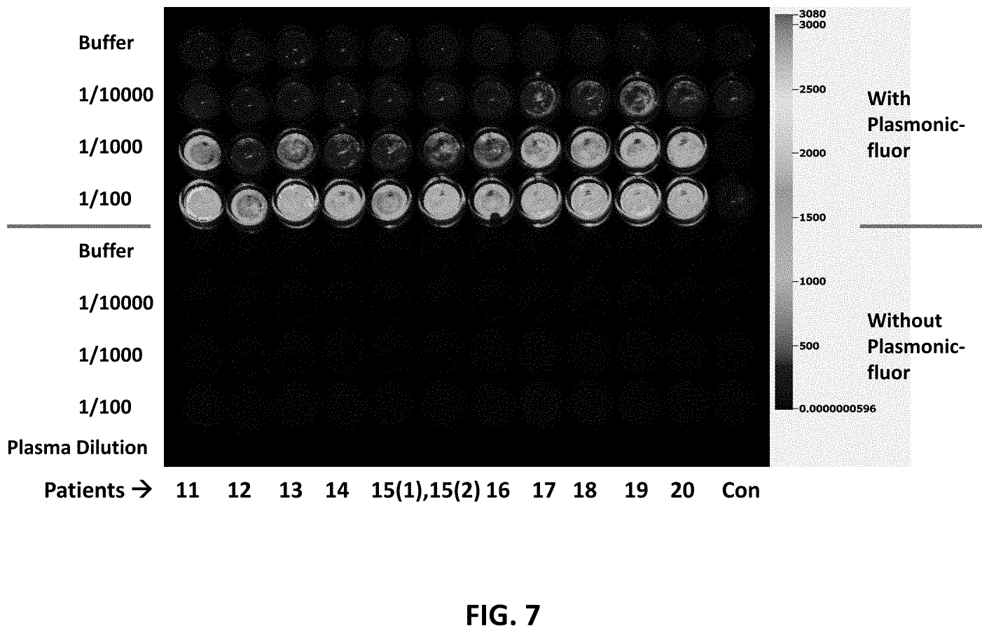

Results: Results are based upon assay of the IgG, IgM and IgA directed to SARS-CoV-2 protein S1 and the RBD within S1 in patient samples 11-20. Eight-well ELISA strips were precoated with recombinant proteins overnight and blocked with bovine serum albumin (BSA). The fast assay consisted of: patient plasma was diluted in assay buffer (1% BSA, 1×PBS, 0.05% Tween-20 (TW20)) and applied to the pre-coated-pre-blocked wells for 5 minutes. Dilutions of biotinylated rabbit anti human IgG (1/1000), IgM (1/1000) or IgA (1/500) in assay buffer were applied for 5 minutes, a 1/2000 dilution of streptavidin-800CW in assay buffer was applied for 5 minutes and finally plasmonic-fluor (0.4 OD at 800 nm) in 1% BSA was applied for 5 minutes, In between each step the wells were washed with 340 ul of 1×PBS containing 0.05% TW20. Wells were also not treated with the plasmonic-fluor after the streptavidin-800CW. The strips were then analyzed by a LI-COR CLx instrument at 800 nm. Typical results are shown in FIGS. 6 and 7. FIG. 6 shows fast assay analysis of the IgG, IgM and IgA of dilutions of patient 19 plasma recognizing SARS-CoV-2 proteins S1, S2, N and the RBD. FIG. 6 shows comparison of fluorescence without (lower half) and with plasmonic-fluor (upper half). In FIG. 6, clearly the addition of the plasmonic-fluor greatly enhances the detection of each immunoglobulin detecting each viral protein. Also, immunoglobulin recognition of N protein is more abundant, hence assays based on this are more sensitive, with a question of specificity within the virus family. FIG. 7 shows detection of IgA to S1 protein in patient plasma. FIG. 7 shows comparison without (lower half) to with plasmonic-fluor (upper half). The fast assay was tested to detect IgA directed to S1 and found that plasmonic-fluor allowed detection of IgA directed towards S1 in all patient plasmas (FIG. 7). Again, plasmonic-fluor greatly enhanced the sensitivity to detect IgA and it is apparent that the relative IgA content of different candidate plasmas for convalescent plasma treatment varies. This was found to be true for the IgG and IgM response of different patient plasmas (not shown).

Methods. The ability of plasmonic-fluor to detect immunoglobulins to SARS-CoV-2 proteins and determination of assay sensitivity was tested with samples of up to 100 patients documented to have SARS-COV-2 infection in the WU 353 cohort. This bolstered the scientific rigor and allow unbiased and thorough testing of the relevant biologic variables of the SARS-COV-2 fast assay. The assay was optimized with respect to well pre-coating conditions (recombinant protein concentration, time of pre-coating and buffer for pre-coating), concentration of biotinylated anti-human immunoglobulin, concentration of streptavidin-fluorophore, and the optical density at 800 or 680 nm of plasmonic-fluor to achieve maximum assay sensitivity within cost analysis. Although all viral proteins were used as pre-coating bait, the assay ultimately focused on S1 and the RBD.

Statistical Analysis. Significance of assay optimization changes were determined to arrive at the best assay possible. Additionally, since the IRB allows collection of patient age, sex, ethnicity and symptoms; analysis was done to determine, if any, correlates of the IgG, IgM or IgA response exist with any of these factors. The titer measure of the S1 response of immunoglobulins was compared to that measured independently to determine conformance.

Based on FIGS. 6 and 7, the plasmonic-fluor confers the requisite sensitivity to reliably diagnose SARS-COV-2 infection in both symptomatic and asymptomatic patients. The assay was independently repeated three times with most patient plasmas to determine inter-assay and in triplicate to determine intra-assay variation. Since patients mount a polyclonal immune response is the sum of the antibodies binding to the S1 or RBD bait, a robust amplification of signal was expected and observed, even with dilution of the plasma. These considerations were used to tune biosensor sensitivity to measure SARS-COV-2 S1 or RBD IgM, IgA and IgG within a concentration range to determine the titer of the antibody response even to a 1/10000 dilution. Since the patient immune response to SARS-COV-2 S1 or RBD is polyclonal, the magnitude of the fluorescent signal of patient plasma is robust even though variable from plasma to plasma. The conditions of the assay are extendable if the CVs of the assay exceed 20% and further for under 10%.

Develop and validate specific biorecognition capability for SARS-CoV-2 immune recognition by epitope mapping of SARS-CoV-2. Identifying IgG, IgM and IgA immune epitopes specific to SARS-CoV-2 adds specificity to ELISA and lateral flow assays that are lacking in existing commercially available assay. Individual immune epitopes in the SARS-COV-2 S1 protein are identifiable using commercially available epitope arrays to further add sensitivity and versatility to nanosensor-based assays.

The biologic function of the adaptive immune system generates antibodies to antigens deemed foreign by the host. This system generates a variety of antibodies each recognizing a small specific region (epitope) of the target antigen; each epitope being recognized by a specific clone of antibody. Due to isotype switching during the genesis of an immune response, an IgM recognizing a specific epitope transitions to an IgA or IgG recognizing that specific epitope. Overall, the immune system generates a polyclonal response to antigens representing the sum of the monoclonal antibodies. As mentioned before, SARS-COV-2 generates N, S1 and S2 proteins early in the infection and the patient, in turn, generate an immune response of IgM, IgA and IgG antibodies after several days (FIG. 5). The S1 and S2 proteins of different corona viruses have sequence diversity as well as sequence similarities. The N proteins of different corona virus members are highly conserved leading to epitope similarity reducing the diagnostic specificity of assays based on N protein-dependent recognition of patient IgM, IgA and/or IgG. However, identification of specific peptides within SARS-COV-2 N uncovers epitopes that discriminate between SARS-COV-2 and other corona virus infections.

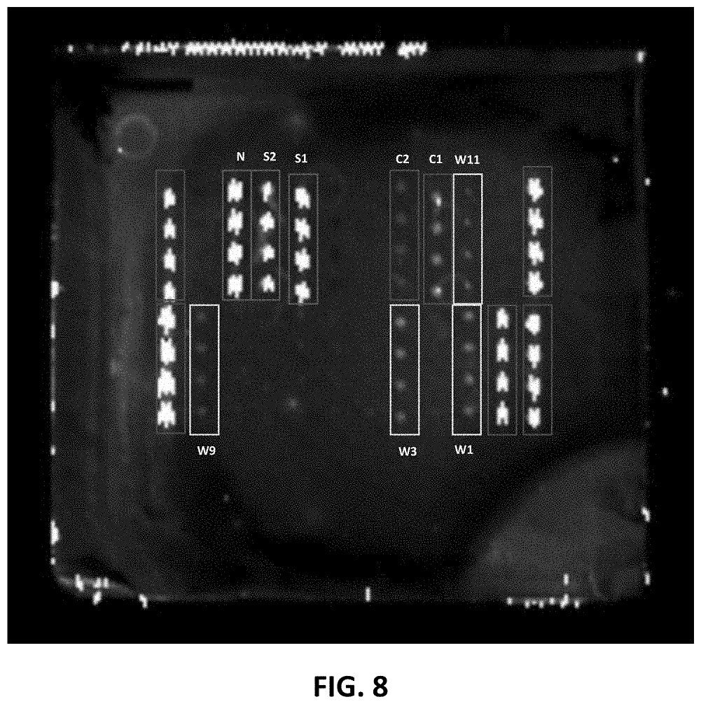

Results. The ability to perform epitope mapping, although on a small scale was shown previously for a monoclonal antibody to an extracellular domain of human aquaporin 1. This technique was applied to define epitopes on SARS-COV-2 S1 protein using commercial microarrays (RayBiotech) with well annotated SARS-CoV-2-infected convalescent sera documented in Aim 1 to bind IgA. These peptide arrays are spotted in quadruplicate with each of 11 SARS-CoV-2-specific peptides, 3 peptides common to SARS-CoV1 and -2, whole SARS-CoV-2 S1, S2 and N proteins and biotinylated bovine serum albumin (BSA) for orientation purposes. Four unique SARS-CoV2 S1 peptides and 2 CoV-1 and -2 common peptides binding patient IgA were identified by incubating the washed slides with biotin-tagged anti-human IgA followed by plasmonic-fluor and visualized by scanning on a LI-COR CLx instrument (FIG. 8). FIG. 8 shows Ray Biotech epitope map to SARS-CoV-2 S1 protein of patient 18 plasma developed for IgA. Blue boxed spots are whole recombinant S1, S2 and N proteins. Yellow boxed spots are SARS-CoV-2 specific peptides while orange boxed spots are both CoV-1 and -2 common peptides. The red boxed spots are biotinylated BSA and immunoglobulins for orientation purposes. This demonstrated that epitope mapping is useful to identify specific epitope peptide sequences to impart assay specificity for the optimized fast assay described herein above.

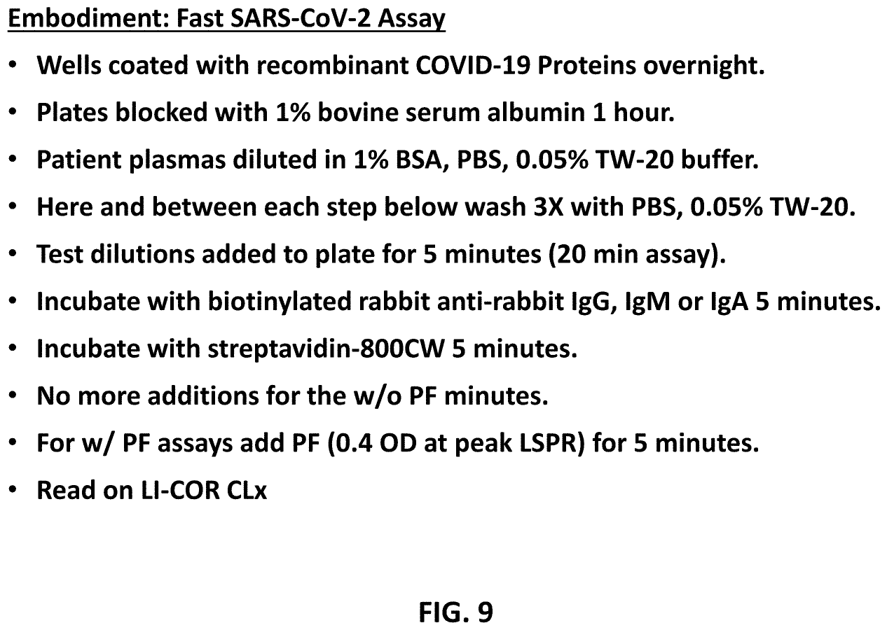

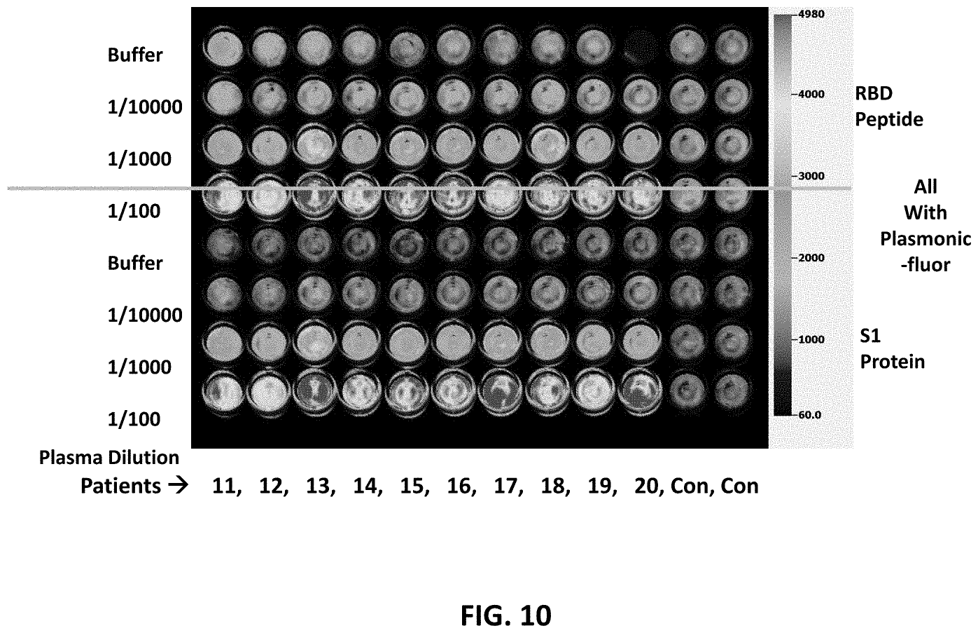

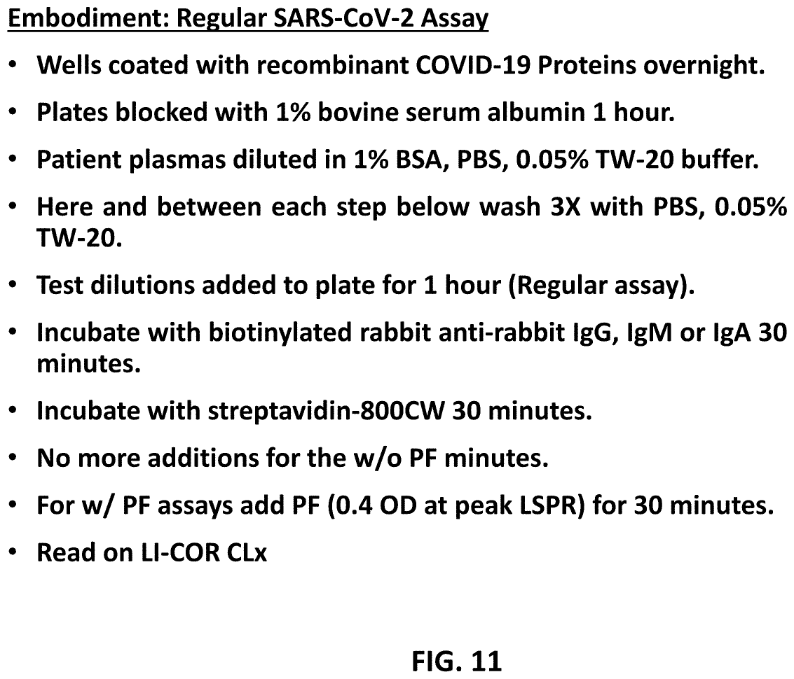

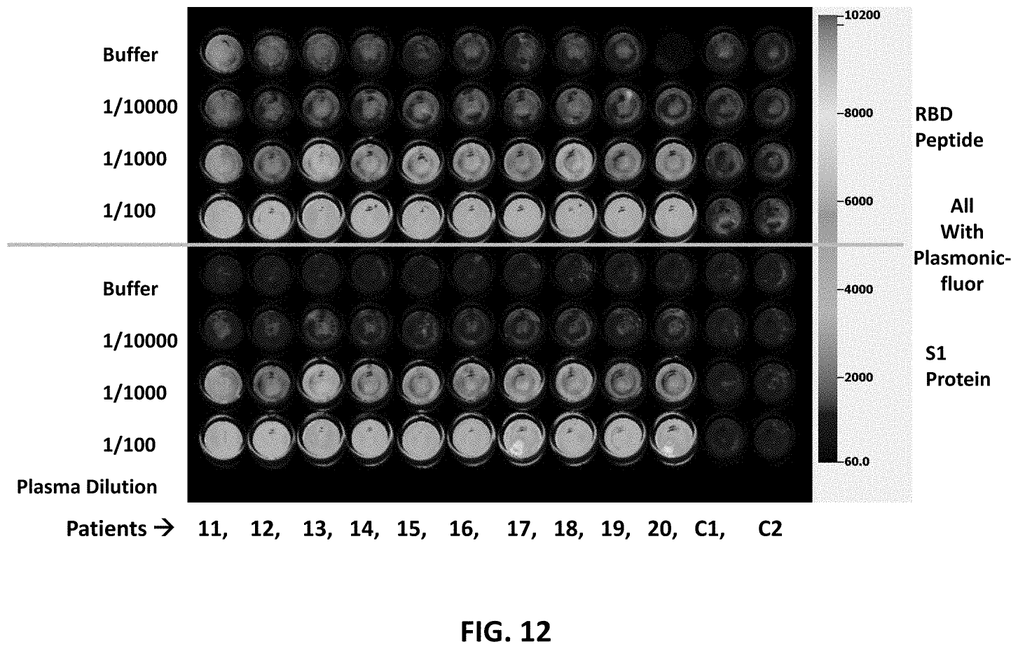

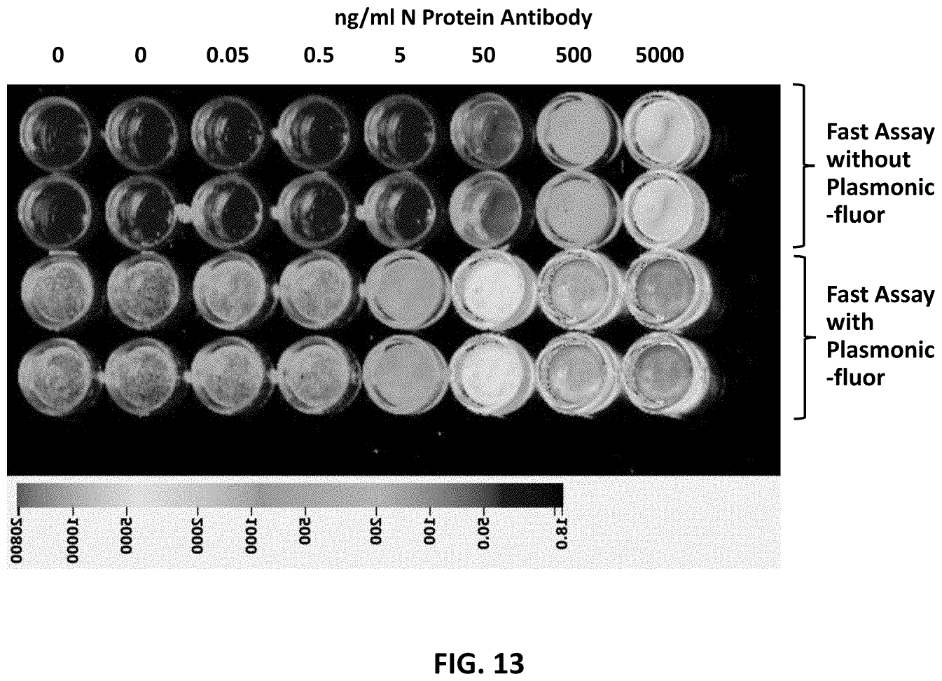

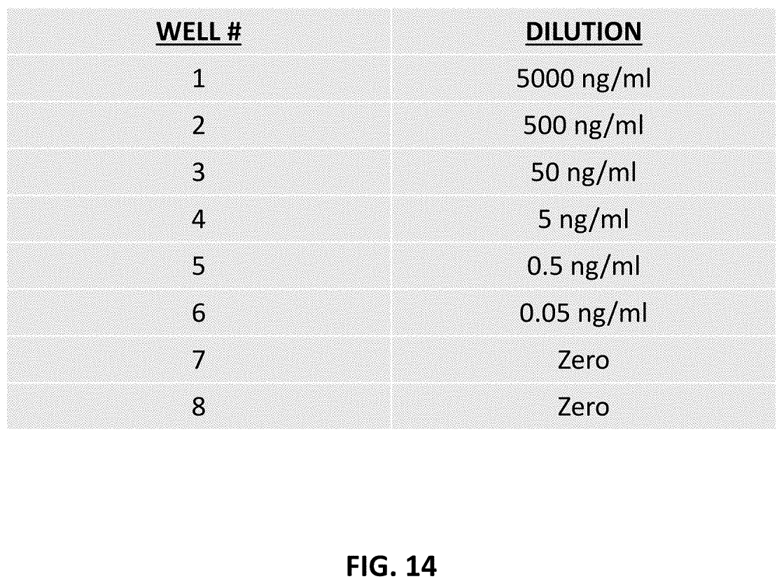

Methods. Commercially available S1 epitope arrays were used to screen convalescent plasmas and identify epitopes recognized individually by the IgG, IgM and IgA present in the candidate plasma using plasmonic-fluor to boost assay sensitivity. Convalescent plasma is actively being used to treat newly infected patients. Based on the screen (FIGS. 9-14) of up to 24 patient (highest determined titers of IgG, IgM and IgA to S1 and RBD) and control plasma (controls pretested to have no S1, S2, N or RBD cross-reactivity) using the Ray Biotech 11 SARS-CoV-2 non-overlapping peptides and 3 SARS-CoV-1 and -2 common peptides, commercially available epitope arrays from PEPperPRINT comprised of 4883 15 amino acid peptides with 13 amino acid overlap in duplicate of all SARS-CoV-2 proteins were utilized to identify as many as possible signature peptides in the SARS-CoV-2 S1, S2, N protein and the RBD of S1, generating a fine-tuned epitope map. Numerous existing assays measure the IgG/IgM response to N protein, the least diverse protein of the corona virus family, thus leading to a lack of specificity. PEPperPRINT arrays have been utilized to map IgG, IgM and Iga epitopes in sera of infected patients. Identifying an epitope or two unique to SARS-CoV-2 N protein reacting to patient IgG, IgM or IgA boosts assay sensitivity, especially that of lateral flow devices while providing the requisite specificity. Also, based on epitope maps, the sites of monoclonal antibodies, cryptic epitopes, or single-domain Camelid antibodies that have therapeutic effects are used to characterize convalescent plasmas containing appropriate neutralizing antibodies for those epitopes for treatment. The dynamic range afforded by the plasmonic-fluor for fluorescent to eye-visible readout accommodates a variety of devices.

Statistical Analysis. The Statistical Analysis Core will be consulted to organize the identified epitopes and determine if certain epitopes correlate with disease severity based on the symptoms as was done in Aim 1. This information may be useful in predicting which epitope peptides are targets for future study.

Outcomes. The mapping identified linear epitopes within the SARS-COV-2 S1 protein and its RBD recognized by the IgMs, IgGs and IgAs of SARS-COV-2-infected patients, though this strategy was less likely to identify conformational or discontinuous epitopes that rely on a special 3-dimensional proximity. If a few viable and specific linear epitopes are found, it confers sufficient specificity to a peptide-baited ELISA. Additionally, these linear epitopes do not directly differentiate between an IgM, IgA or IgG. Narrowing the time from infection (early IgM vs. later IgG and IgA) helps to determine the circumstance of exposure, particularly in asymptomatic individuals, to reconstruct patterns of SARS-COV-2 spread or the circumstance of infection to allow contact tracing. This extra specificity when added to the sensitivity is crucial to detecting individuals who are asymptomatic and in population screening to determine the true extent of infection in the community.

Creation of rapid, ultra-sensitive and specific assays for SARS-CoV-2 ELISAs and lateral flow devices. Identification of peptide epitopes specific for SARS-CoV-2 S1, S2, N and the RBD can be used to prepare peptide baits on gold nano-islands decorating the wells of standard 96-well ELISA plates for high throughput screening of candidate convalescent plasmas or for population screening. Appropriate peptides can be synthesized with additional glycine residues culminating in a cysteine to couple to the gold surface through stable Au—S bonds. Similarly prepared 8-well strips in the 96-well format can be used to spot screen plasmas. Additionally, lateral flow devices using a mix of SARS-CoV-2 specific peptide epitopes can be used for a visual output based on the versatility of the plasmonic-fluors to provide both a fluorescent and a visible readout. Assays for cTnI using peptide baited gold nanostructures have been developed and the shift in localized surface plasmon resonance has been measured. Additionally, generic corona virus epitopes may be multiplex with SARS-COV-2-specific nanosensors along with pan-corona virus peptide sensors to broaden the serologic diagnostic ability of the assay. Since members of the corona virus family have common symptomology and thrive in similar circumstances, a multiplexed broad spectrum nanobiosensor would be advantageous to determine the precise infective source.

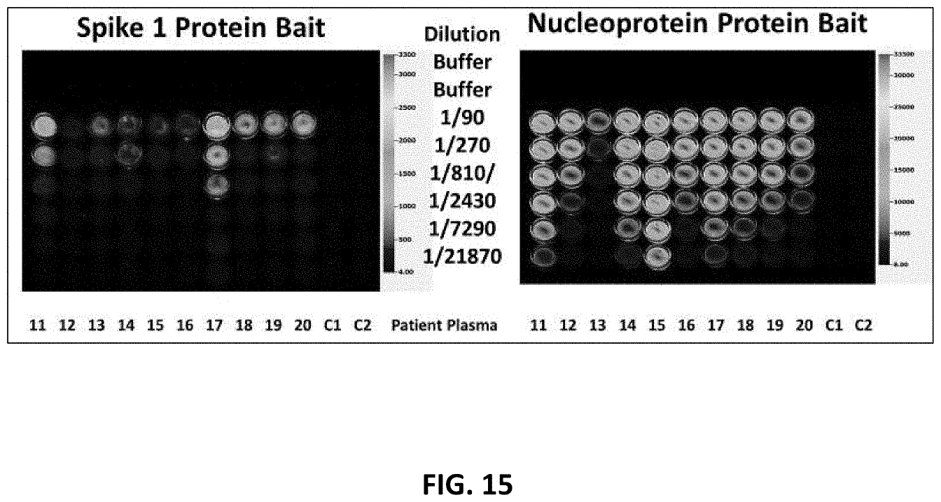

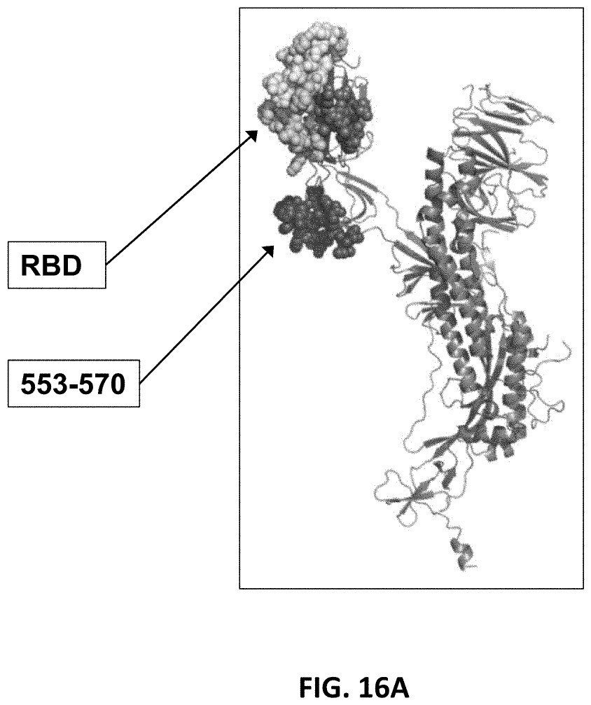

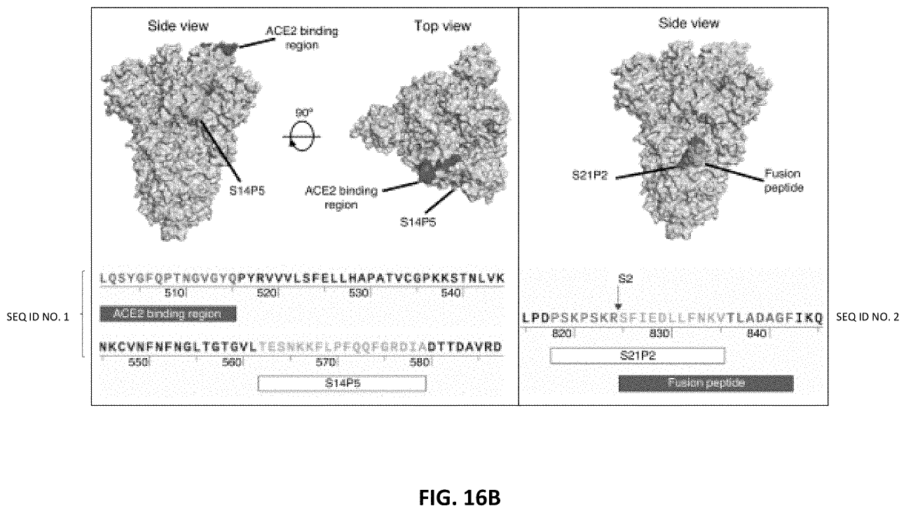

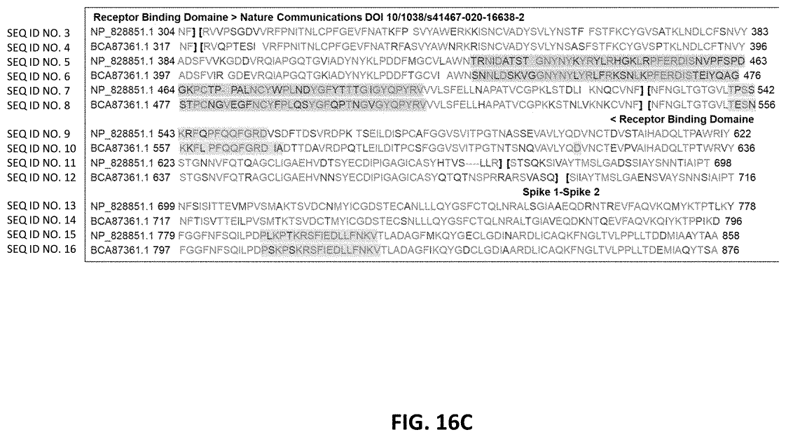

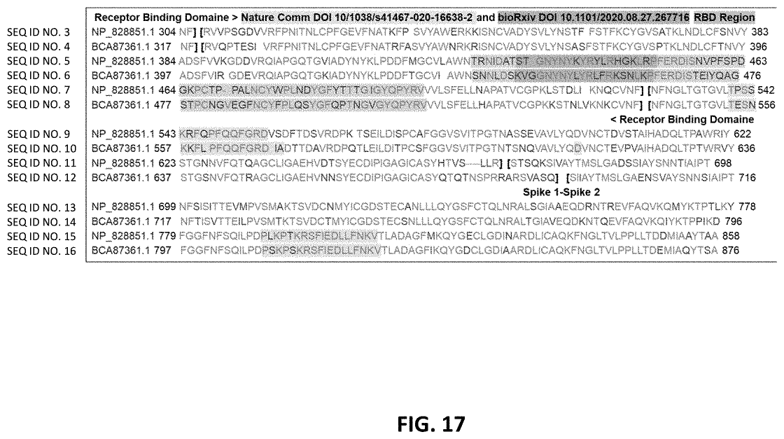

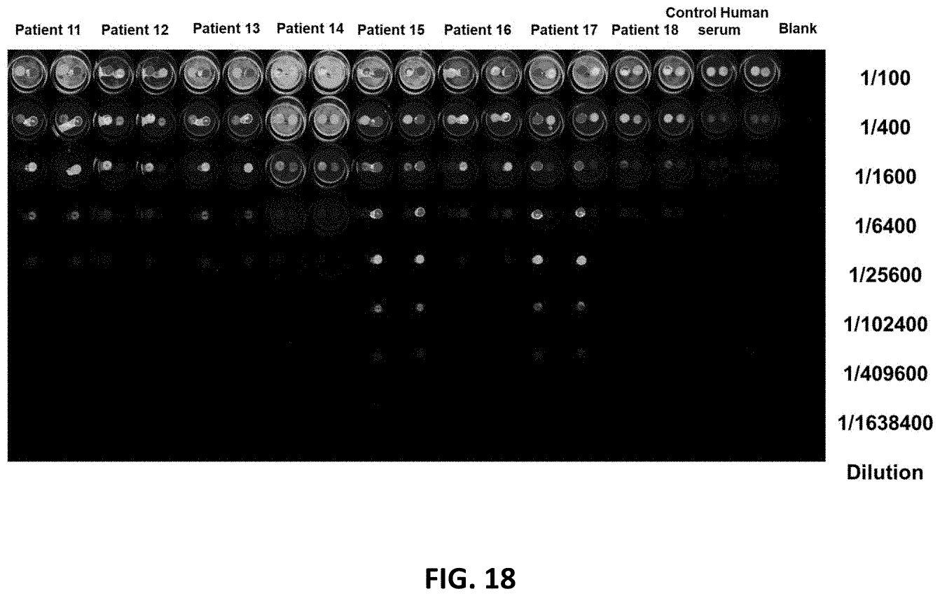

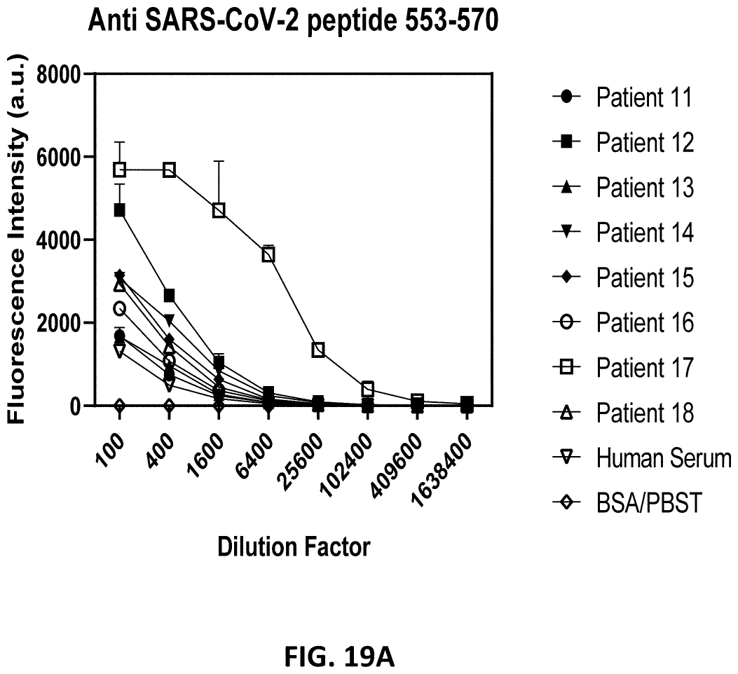

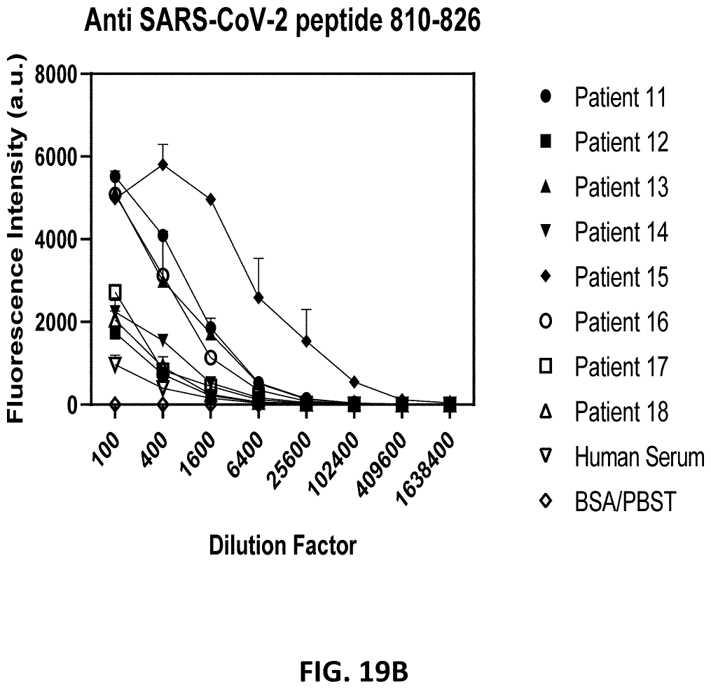

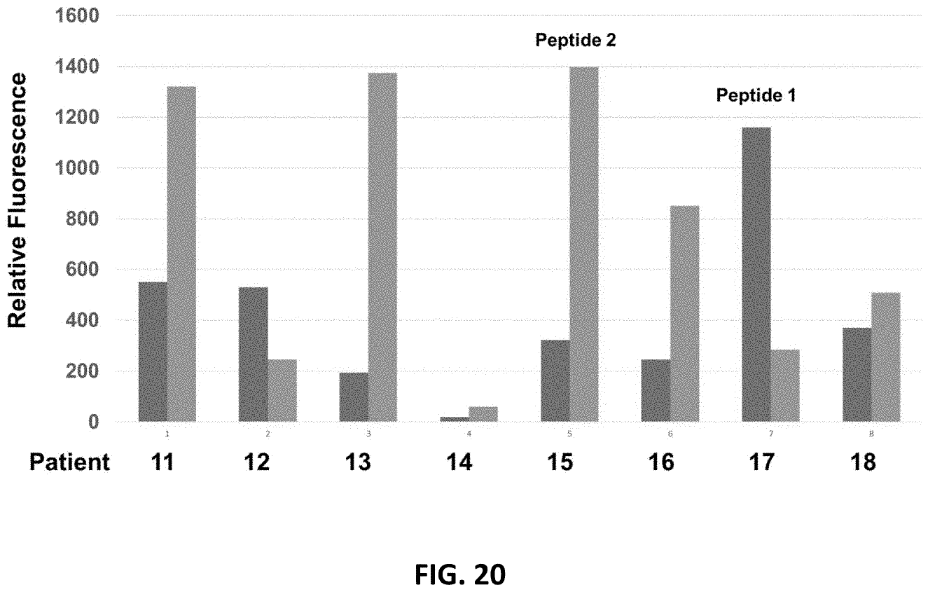

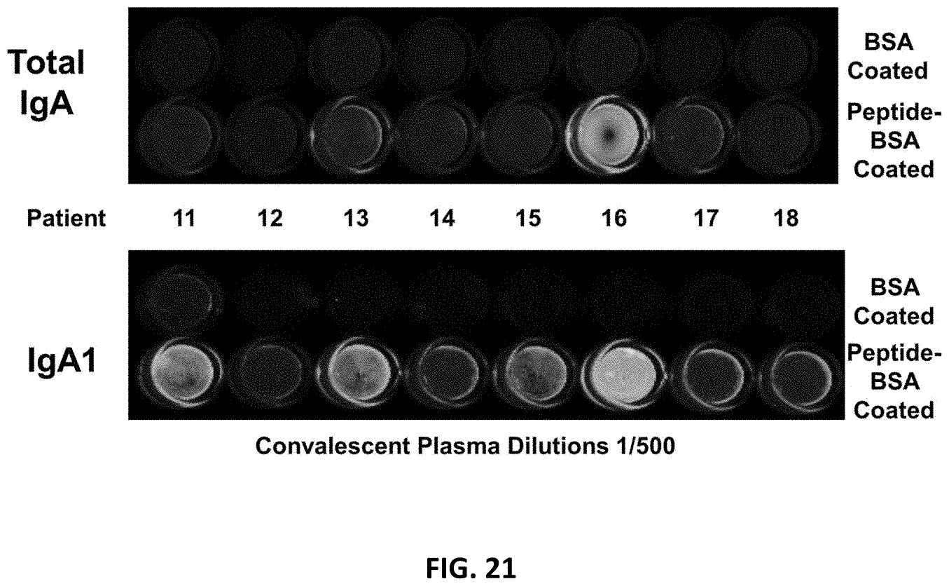

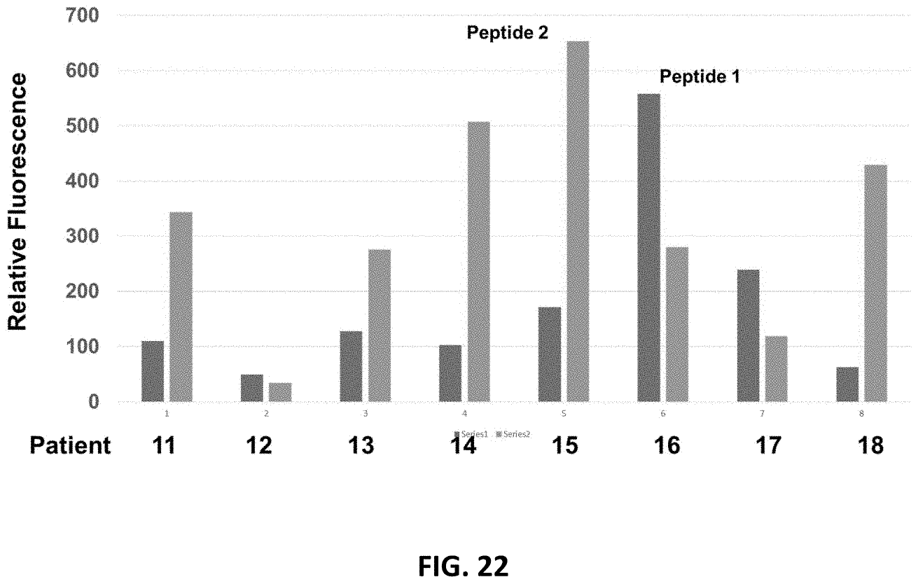

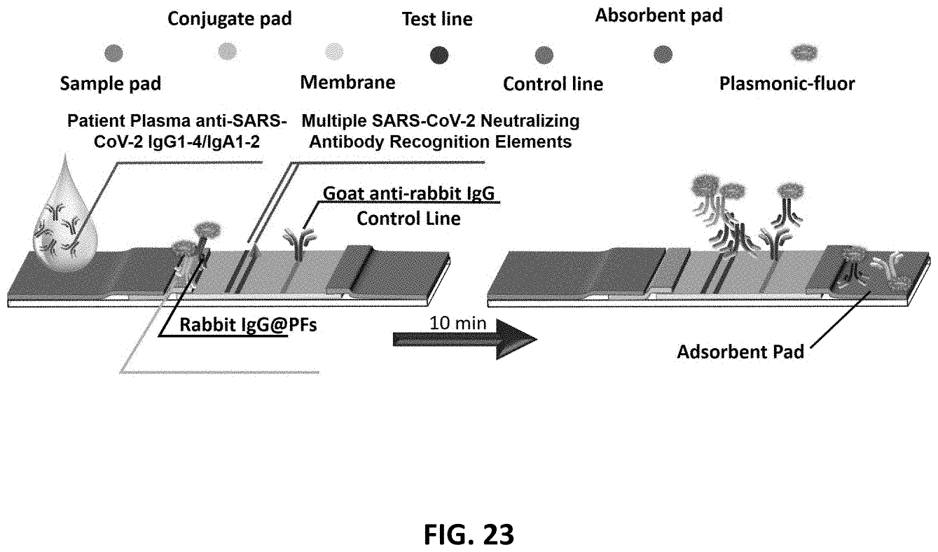

Plasmonic-fluor provides significant improvements for measuring the immune response to SARS-CoV-2 infection. Measurements in accordance with the present disclosure include amplification of single-well analyte-immunoglobulin detection of the polyclonal response to SARS-CoV-2 infection (FIGS. 15, 20-22) and detecting a monoclonal response to single linear neutralizing epitopes (FIGS. 16(A-C), 17) within SARS-CoV-2 spike protein in a single well ELISA assay or a multiplexed well ELISA assay (FIGS. 18, 19(A-B)). This involves having peptides made of the suspected linear epitopes, linking them to bovine serum albumin as a scaffold and using these BSA-peptide conjugates as bait to assess convalescent plasma. As disclosed herein, a multiplexed lateral flow assay (FIG. 23) incorporates these improved assays in to measure for several monoclonal neutralizing antibodies to select suitable convalescent plasmas for patient treatment. Some patient plasmas have autoantibodies to interleukins and interferons that severely compromise the ability of those patients to fight disease progression. As disclosed herein, analysis of these autoantibodies is included in the multiplexed lateral flow format.

### Plasmonically-Enhanced Ultrasensitive Epitope Specific Serologic Assay for COVID-19

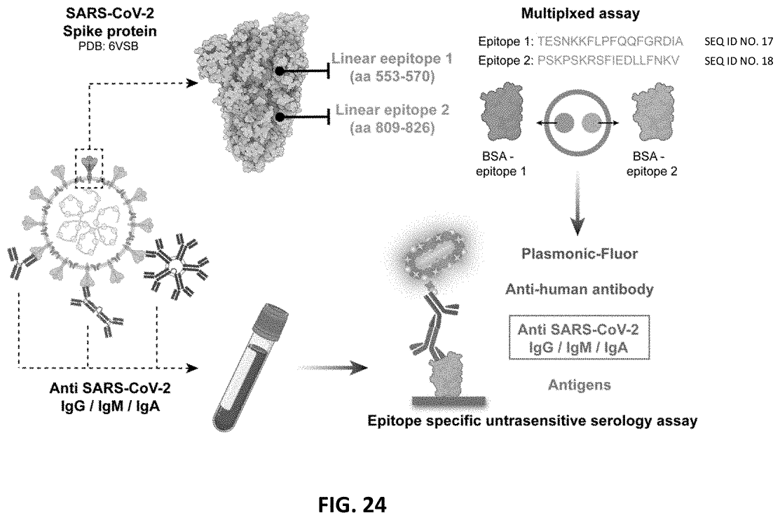

Integration of a plasmonic-fluor, an ultrabright fluorescent nanolabel, was demonstrated with SARS-CoV-2 serology assays to achieve the ultrasensitive detection of epitope-specific antibody isotype and subclass in both a microtiter whole well format and a spatially-multiplexed manner measuring two different epitopes within a single microtiter well. Contrary to the conventional serological tests relying on whole protein or large protein domains, BSA-peptide was employed encoding specific epitope sequences from SARS-CoV-2 spike protein as the antigen and plasmonic-fluor as an ultrabright and highly specific fluorescent nanolabel. Plasmonic-fluor achieves more than 6000 times brighter florescence signal compared to the conventional fluorophores. Plasmonic-fluor improved the sensitivity up to three orders of magnitude for numerous bioanalytical techniques, including immunomicroarrays, fluorescence linked immunosorbent assay (FLISA), bead-based fluoroimmunoassays and flow cytometry. As described herein, application of plasmonic-fluor demonstrated results in an ultrasensitive serology assay, employable for the detection and quantification of SARS-CoV-2 epitope-specific antibodies in convalescent patient plasma in both biomedical research and clinical diagnosis (FIG. 24).

Results and Discussion

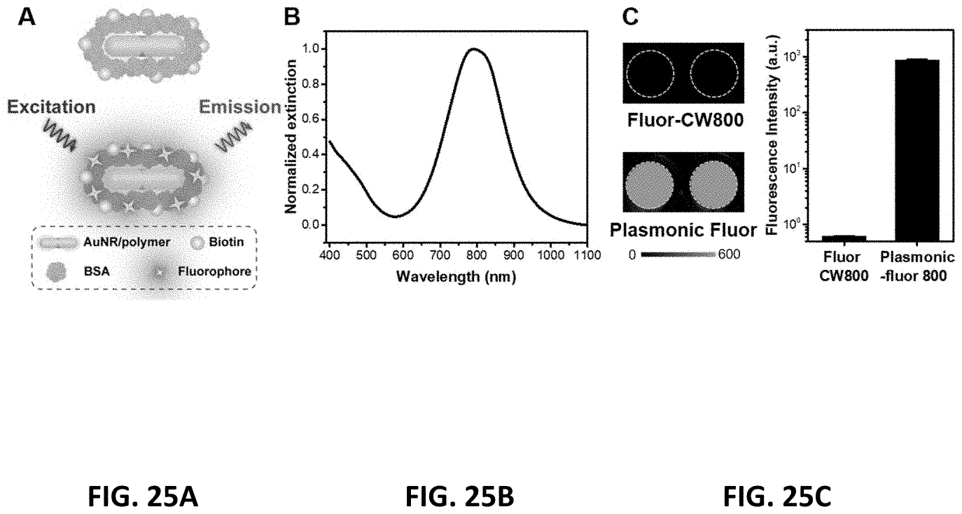

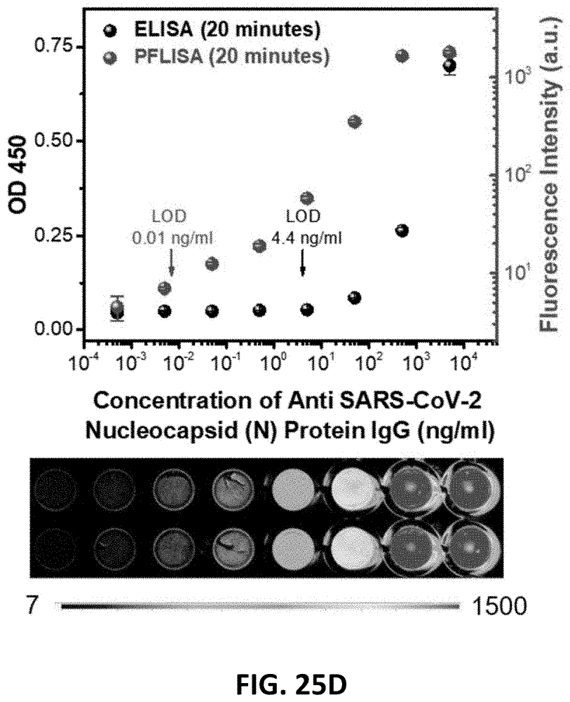

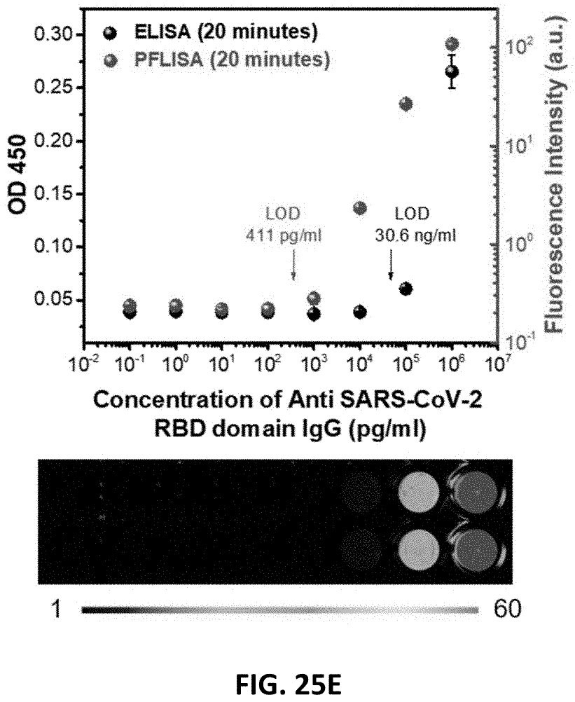

Conventional serological tests detect antibodies via a sandwich enzyme-linked immunosorbent assay (ELISA). The enzymatic reaction results in the formation of soluble colored products in an antibody concentration-dependent manner. While routinely employed, this approach is not suitable for the fast, sensitive, and multiplexed detection of epitope-specific antibody, due to (1) relatively low sensitivity, making the fast quantification of low abundant antibody challenging, and (2) the soluble nature of the colored product, precluding the possible spatially-multiplexed detection. Therefore, existing technologies for COVID-19 are limited to the detection of antibodies against the whole S protein, RBD domains or N proteins, which only provide incomplete information with possible high false positive rates. To overcome this challenge, a fluorescence-linked immunosorbent assay (FLISA) was used that relies on plasmonic-fluor as an ultrabright and highly specific fluorescent label (FIG. 25A). Plasmonic-fluor is comprised of a gold nanorod (AuNR) coated with fluorophores (800CW) and a universal biorecognition elements (biotin).17 Bovine serum albumin (BSA) is employed as a scaffold to assemble all functional elements and to minimize non-specific protein binding. A siloxane copolymer spacer layer between the AuNRs and the fluorophores is employed to avoid metal-induced fluorescence quenching. The relatively narrow longitudinal localized surface plasmon resonance (LSPR) wavelength of the plasmonic-fluors indicated the colloidal stability of the nanolabels (FIG. 25B). Binding of the plasmonic-flour-800CW to streptavidin-CW800 coated on microtiter plates resulted in a nearly 1500-fold enhancement of ensemble fluorescence intensity (FIG. 25C).

To investigate the applicability of plasmonic-fluor as an ultrabright nanolabel in a fast SARS-CoV-2 serological assay, anti SARS-CoV-2 N or Se protein antibodies were used as analytes and the entire assay completed within 20 minutes (incubation time in each step is 5 minutes). Conventional SARS-CoV-2 antibody ELISA involves a standard sandwich immunoassay format: immobilization of the antigen (recombinant S or N proteins) on the bottom of microtiter plate, capture of target antibodies, recognition and binding of biotinylated anti-human antibody and exposure to the streptavidin-HRP. In contrast to conventional antibody ELISA, plasmonic-fluor linked immunosorbent assay (p-FLISA) involves the use of plasmonic-fluor as the label (FIG. 24). To determine the sensitivity and limit of detection (LOD, defined as mean+3σ of the blank) of ELISA and p-FLISA in detecting the target antibodies, serially diluted anti SARS-CoV-2 N protein IgG solutions of known concentration (1 to 106 pg/ml) were used as standards. In this 20-minute assay, LOD of conventional p-FLISA was found to be 10 pg/ml, 440-fold lower than that of the ELISA (4.4 ng/ml) (FIG. 25D). Similarly, the LOD of p-FLISA for anti-S protein RBD domain IgG was measured to be nearly 411 pg/ml, which is 80-fold lower than that of conventional ELISA (30.6 ng/ml) (FIG. 25E).

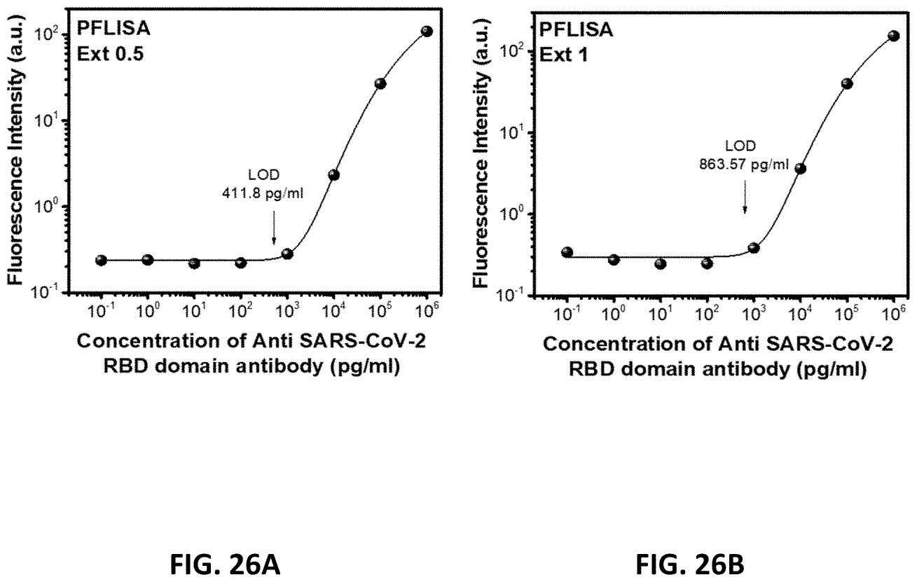

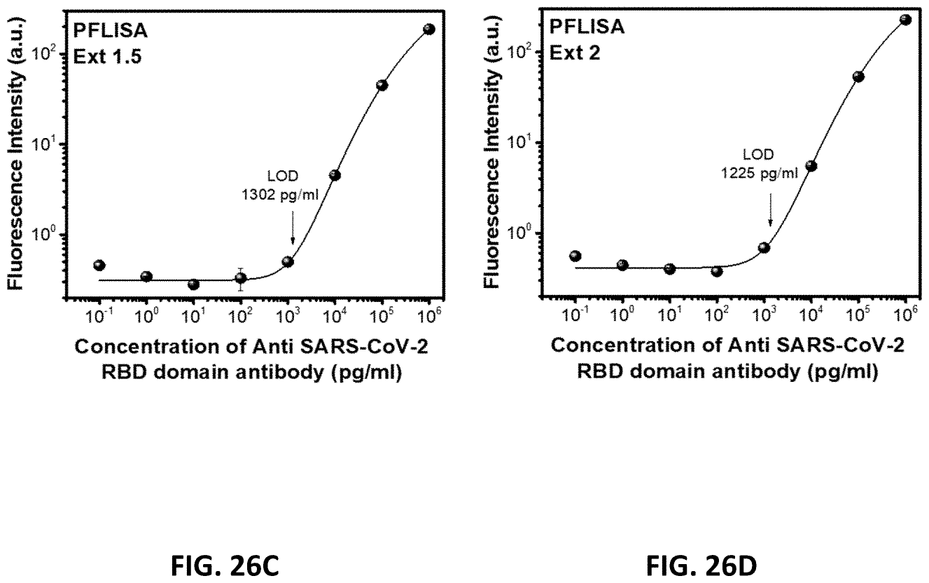

The remarkable brightness of plasmonic-fluor, while greatly improving the efficiency and detection limit of the assay, resulted in higher background signal with even low non-specific binding, consequently affecting the specificity. To test the signal-to-noise ratio before and after applying plasmonic-fluor, BSA coated wells were employed as blank and streptavidin-CW800 coated wells as samples. The background signal increased with an increase in the concentration of plasmonic-fluor. However, the signal-to-noise ratio with plasmonic-fluor (OD of 0.5) is more than 170-fold higher than that of conventional fluorophore. The LOD of p-FLISA for SARS-CoV-2 S protein RBD domain antibody was measured at 411 pg/ml, 864 pg/ml, 1302 pg/ml and 1225 pg/ml corresponding to plasmonic fluor OD values of 0.5, 1, 1.5 and 2, respectively, which are all substantially lower than that of antibody ELISA (30.6 ng/ml) (FIG. 26(A-D)). High concentration of plasmonic-fluor resulted in a slight increase in the background and, as a consequence, compromised the LOD of the assay. The plasmonic fluor with OD 0.5 was employed in the following experiments.

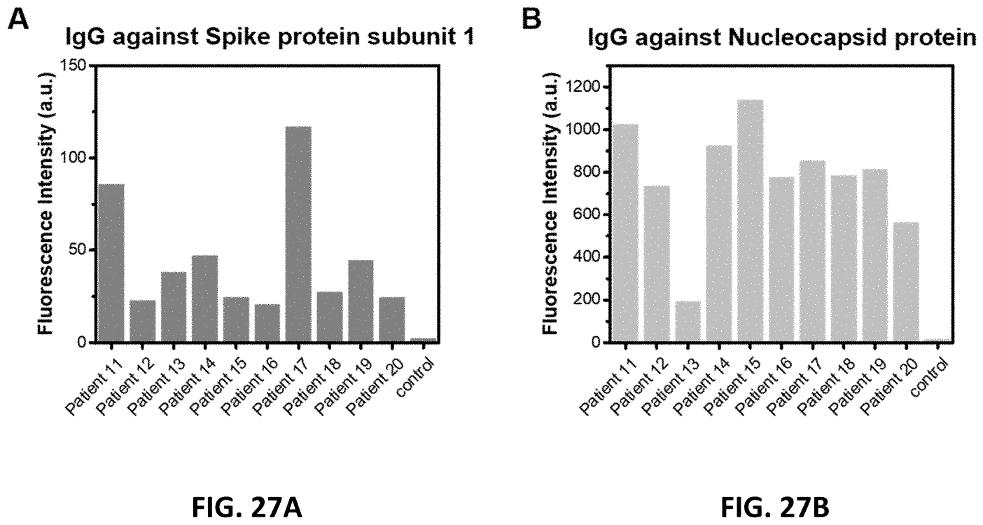

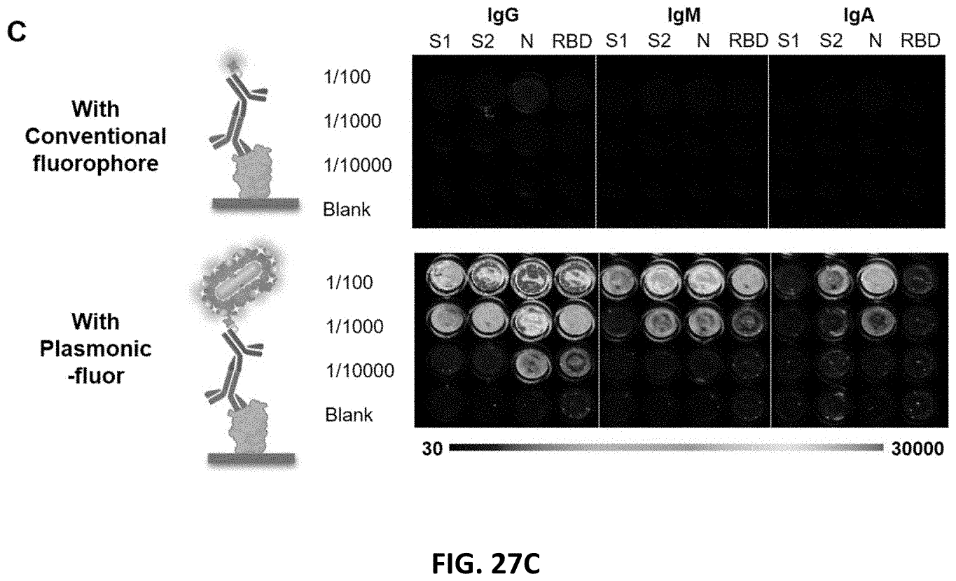

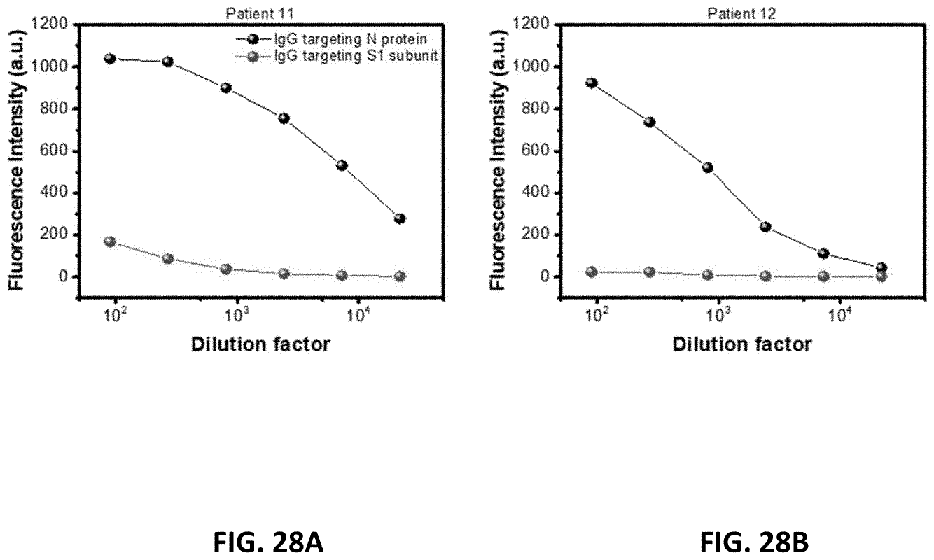

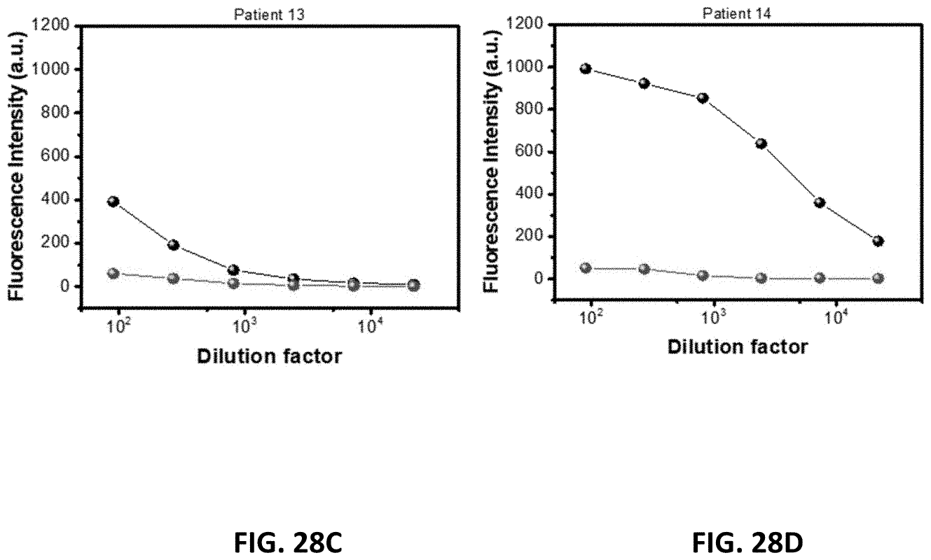

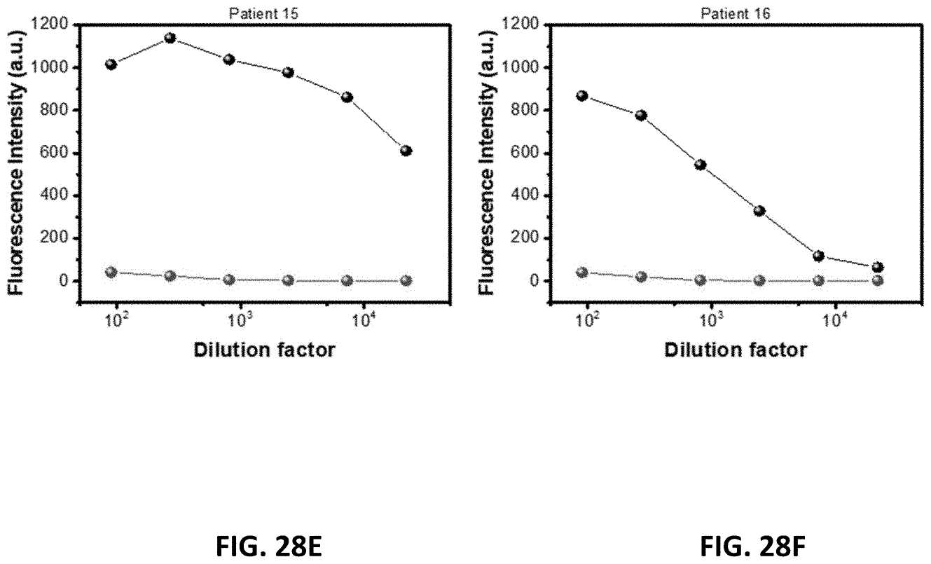

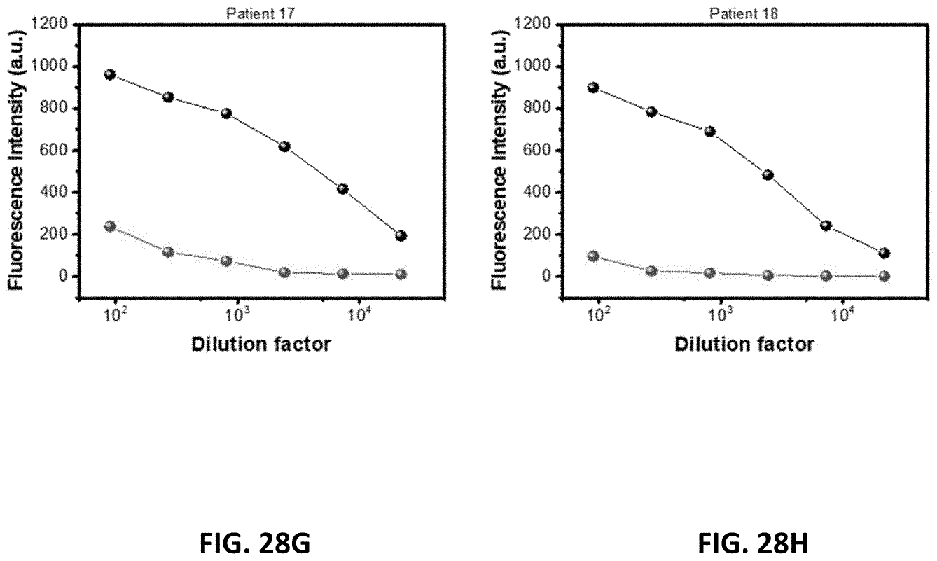

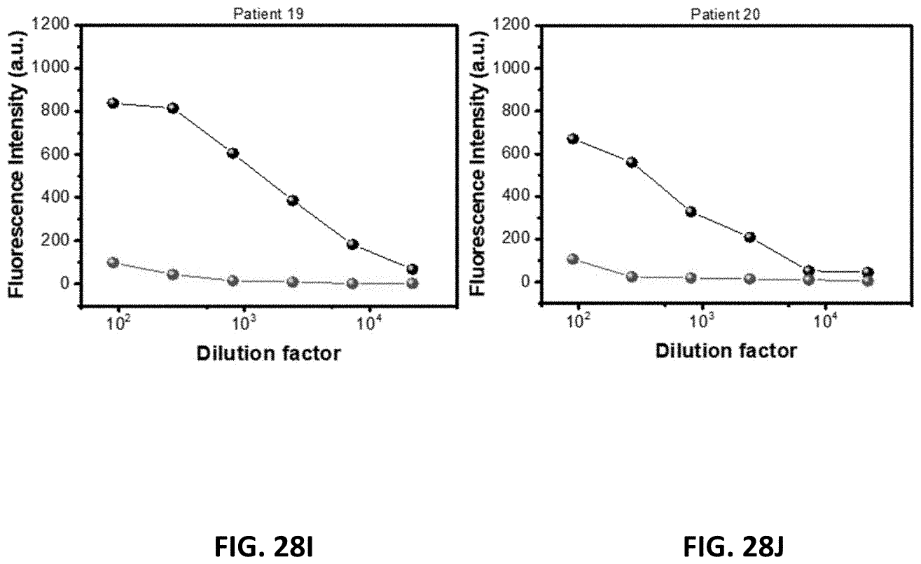

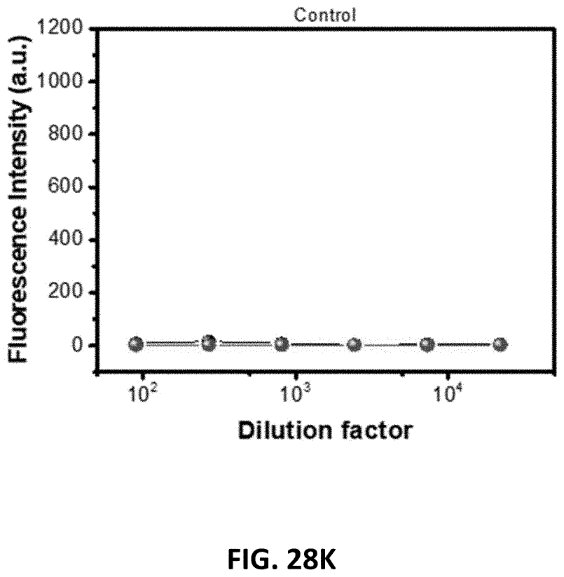

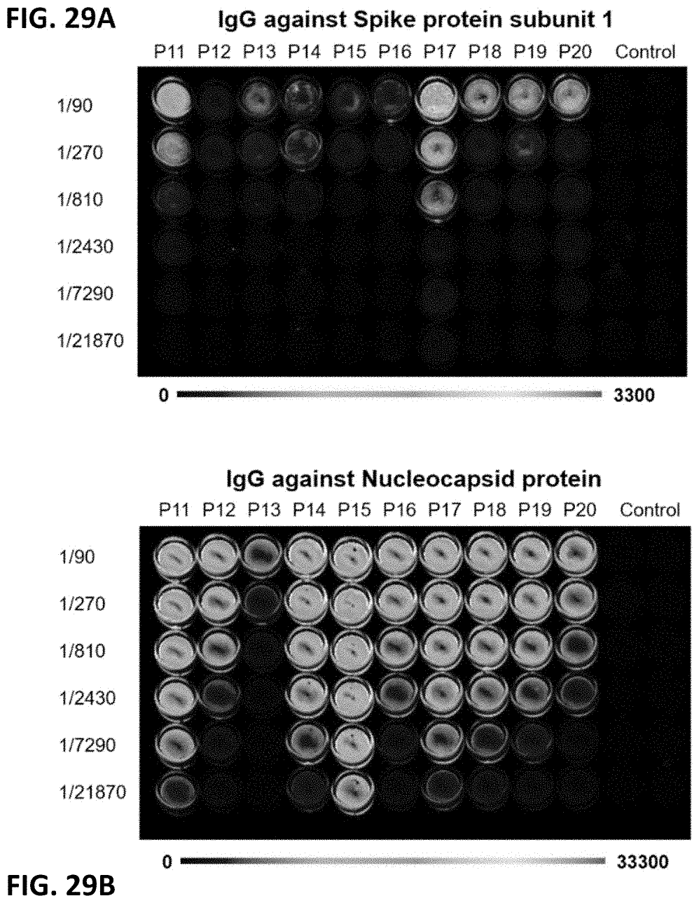

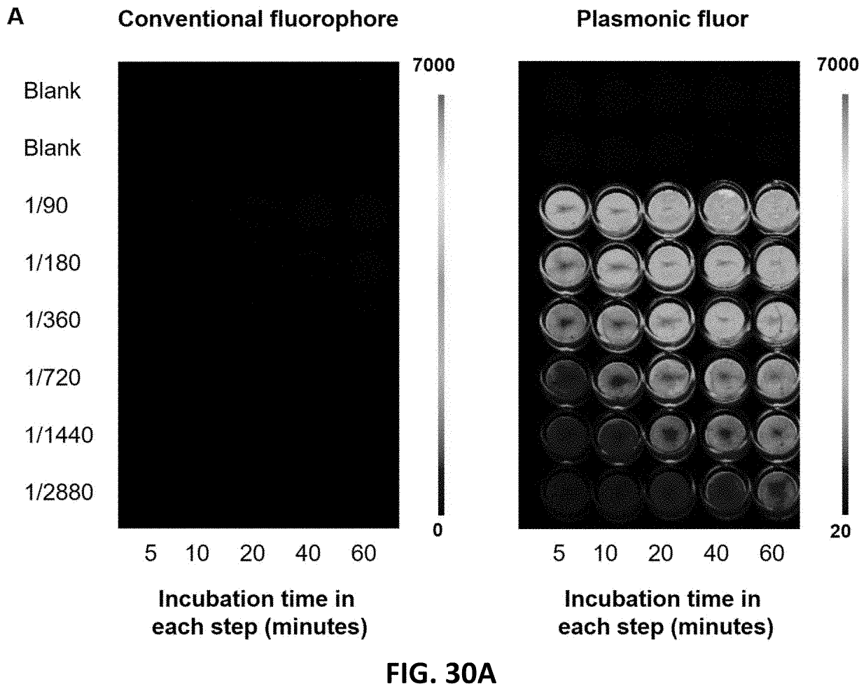

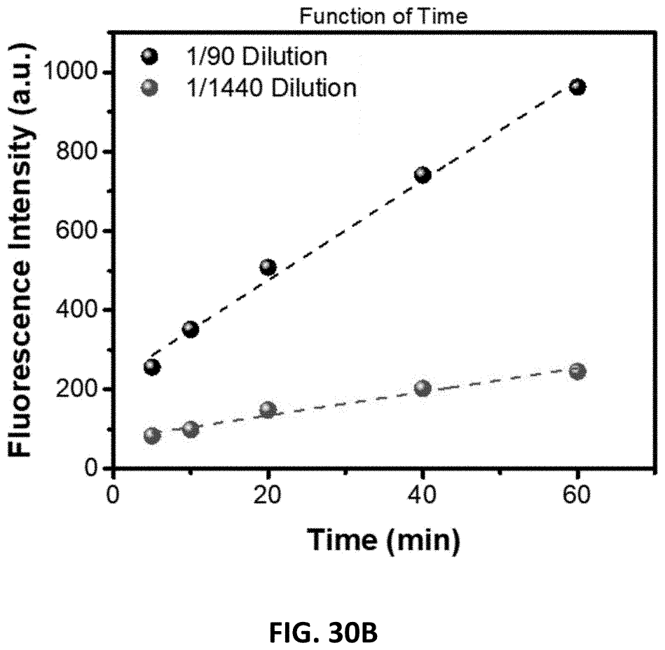

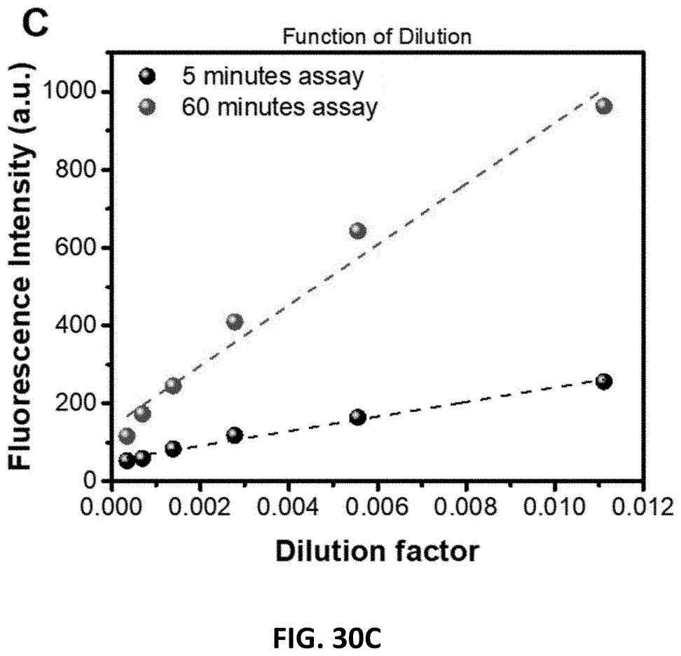

To validate the performance of p-FLISA, SARS-CoV-2 antibodies were detected in 10 plasma samples from PCR-confirmed COVID-19 positive patients collected after their recovery and a healthy control sample acquired before COVID-19 outbreak. Targeting S protein subunit 1 and N protein were first measured and analyzed IgG using 20-minute p-FLISA. Fluorescence intensity corresponding to convalescent plasma samples is much higher than that of the healthy control sample (FIGS. 27A, 27B and 28(A-K)). The levels of anti N protein IgG are significantly higher compared to anti-S protein subunit 1 IgG across all patients. Notably, owing to the high sensitivity of 20-min p-FLISA, anti N protein IgG in 9 out of 10 patient samples are still detectable even after more than 20,000-fold dilution (FIG. 28(A-K) and 29(A-B)). To further compare with the conventional FLISA, antibodies levels were measured and compared in serially diluted convalescent plasma from patient 13 using antibody FLISA and p-FLISA with different incubation durations in each step, from 5 minutes to 60 minutes. Fluorescence intensity obtained with plasmonic-fluor are nearly 1900±200-fold higher compared to conventional fluorophore for different dilutions of plasma at each time point (FIG. 30A). Moreover, fluorescence intensity in both assays demonstrate excellent linearity with assay time and dilution factor, suggesting accurate detection of IgG in patient plasma with as short as 5 minutes of incubation time with diluted patient plasma (FIGS. 30B and 30C).

To evaluate the detection ability of multiple antibody isotypes, plasma from patient 19 was employed as a representative sample and measured isotype levels against viral antigens including S 1, S 2, and RBD domain of S protein and the N protein using 20-minute FLISA and p-FLISA. Fluorescence intensity obtained after applying plasmonic-fluor successfully revealed the existence all isotypes targeting different viral antigens even at more than 1000-fold dilution (FIG. 27C). Conversely, conventional FLISA exhibited negligible fluorescence signals for all isotypes, except for the most robust immune response, IgG targeting N protein at 100-fold dilution (lowest dilution factor).

The antigenic drift and consequent escape from current therapeutic interventions have been the major concerns of COVID-19. For example, mutations including the deletion in the NTD of S1 protein18 have been reported to alter activity of neutralizing antibody. Previously, two linear epitopes on spike protein of SARS-CoV-2 prototypes were found to elicit potent antibodies in convalescent plasma upon the infection, mostly for IgGs, but also for IgA. Epitope 1 (aa 553-570) nearby the receptor binding domain is specific to SARS-CoV-2, while epitope 2 (aa 809-826) encompassing the fusion peptide is highly conserved in generic coronavirus (FIG. 24). Antibody depletion assays demonstrated that antibodies recognizing these two epitopes significantly contribute to patient neutralization capacities for COVID-19. Therefore, a simple and rapid epitope specific serology assay is instrumental in answering vaccine efficacy, longevity of effective immunization and the natural coverage of vaccine-naive but infected individuals, particularly in case of SARS-CoV-2 variants by simply incorporating epitopes of the identified or future identified mutations.

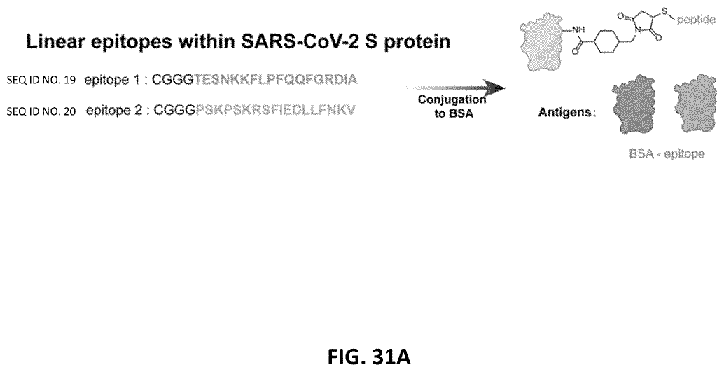

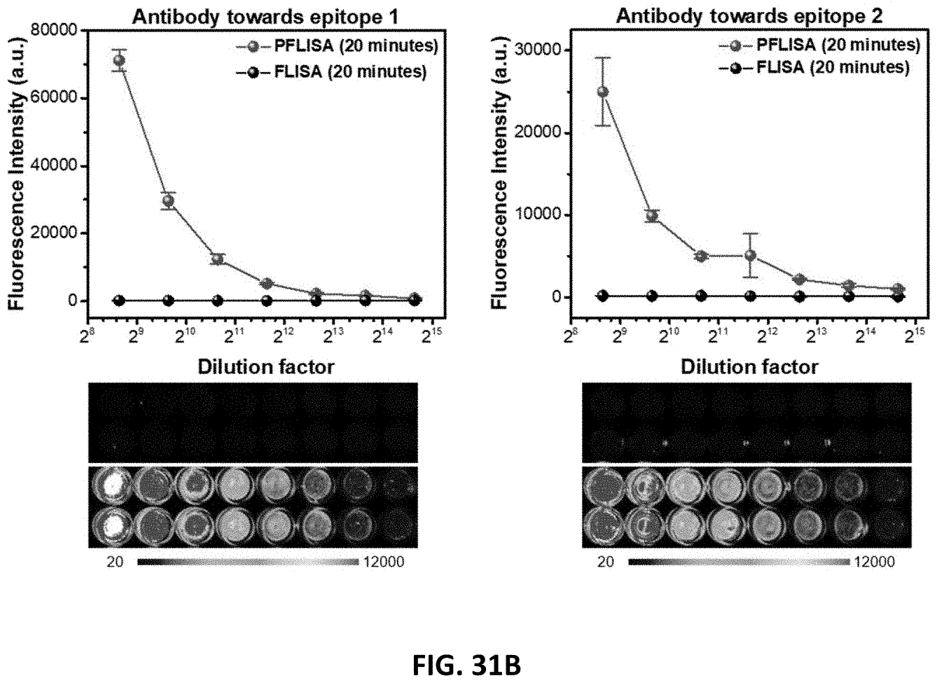

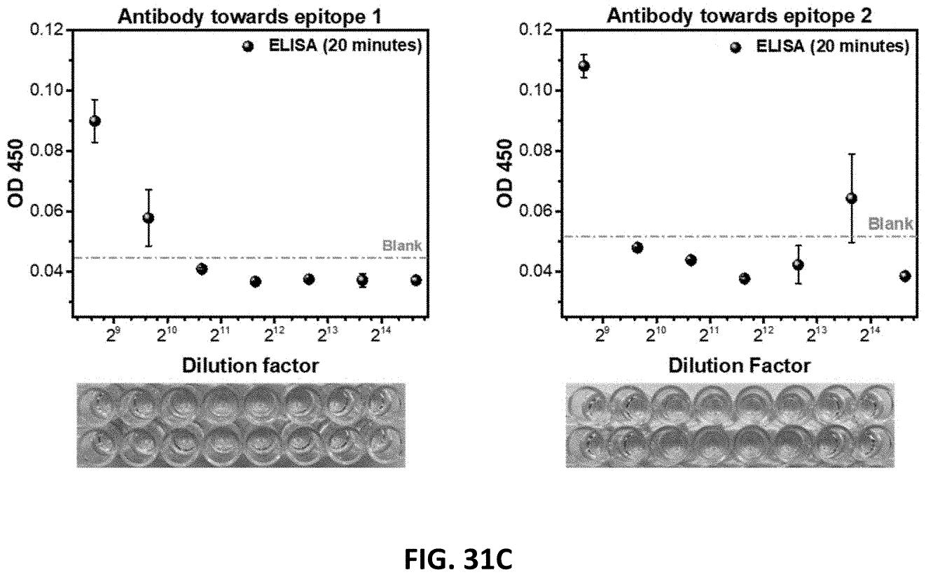

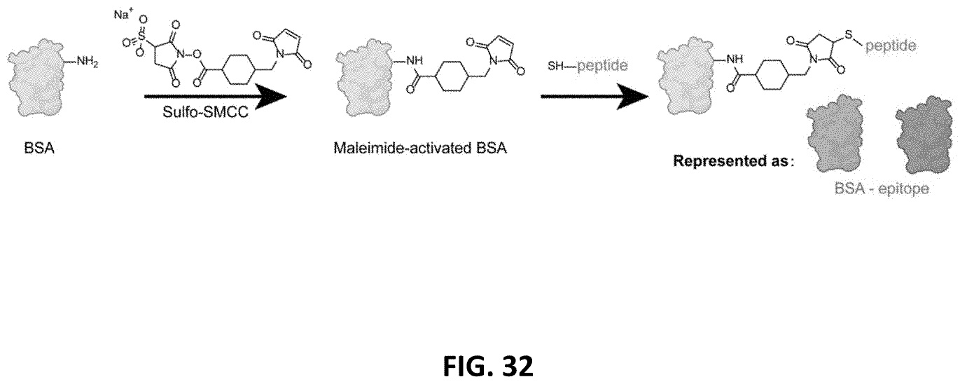

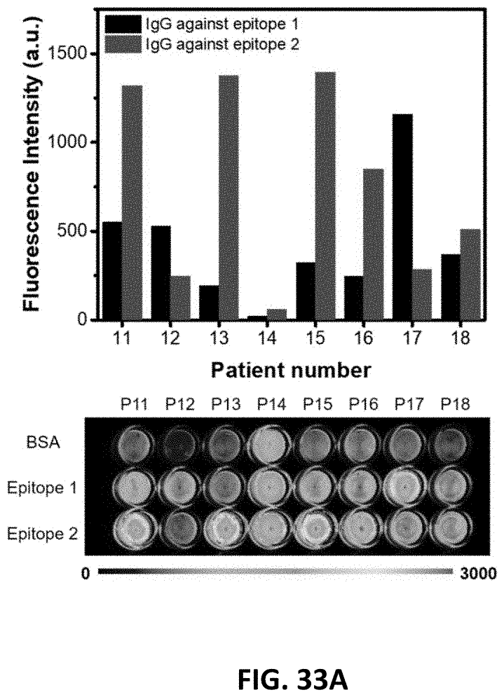

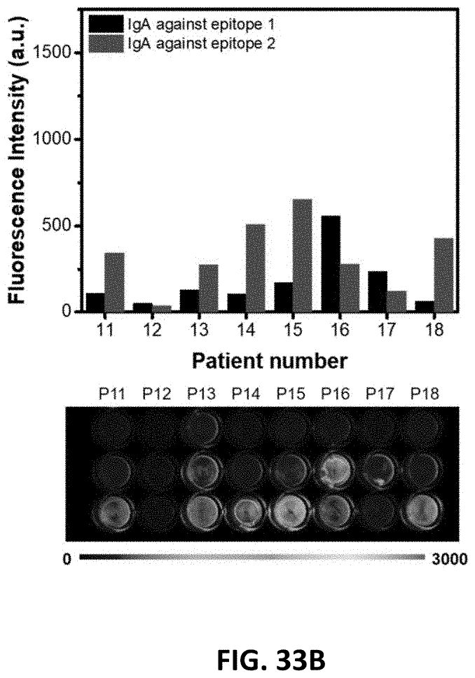

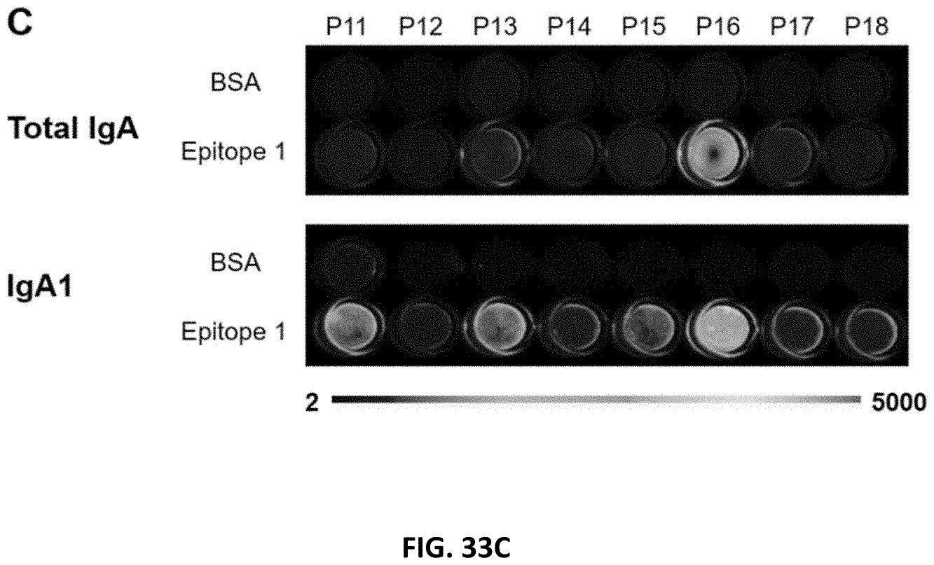

To detect epitope specific SARS-CoV-2 antibodies, BSA-peptides were employed as the capture elements, instead of pristine whole S or N protein. To conjugate SARS-CoV-2 specific peptides to BSA, the peptide comprised of epitope sequence was appended with a triglycine spacer and a cysteine residue at the N terminal. These peptides were covalently bound on BSA via a bifunctional crosslinker with N-hydroxysuccinimide (NETS) and maleimide group at either ends, reactive to amines and sulfhydryls respectively (FIGS. 31A and 32). To compare the ability of 20-minute antibody FLISA and p-FLISA in the detection of epitope-specific IgG, serial diluted convalescent plasma was employed as sample. The fluorescence signals obtained with conventional fluorophores corresponding to both epitopes were identically weak and approaching the background noise, while the ones after applying plasmonic fluor exhibited an excellent dilution-dependent curve, with stronger immune response towards epitope 1 (FIG. 31B). In contrast, the colorimetric signals acquired from conventional antibody ELISA exhibited a large deviation and merely demonstrate discernable signal at the lowest dilution factor (FIG. 31C). To compare the immune response of different immunoglobulin isotypes and even subclasses, the amount of epitope-specific IgG, IgA, and a unique subclass, IgA1, was evaluated from 8 different patient samples through 20-minute p-FLISA PFLISA. Notably, the obtained fluorescence signal demonstrates that the overall immunoglobulin response of IgG is stronger than IgA in convalescent plasma (FIGS. 33A and 33B). Fluorescence signals for IgG indicate different profile of antibodies generated against these two epitopes, where patient 11, 13, 15, 16 and 18 demonstrate higher IgG amount targeting epitope 2 (FIG. 33A). On the other hand, fluorescence signals obtained for IgA suggest that epitope 2 specific antibody is dominant in patient 11, 14, 15, and 18 (FIG. 33B). Specifically, IgG in patient 14 exhibited high background binding to albumin, while low non-specific binding in case of IgA. P-FLISA also enabled the detection of epitope specific IgA1, a unique subclass of antibody, correlating with the amount of total IgA but with even lower background signal (FIG. 33C).

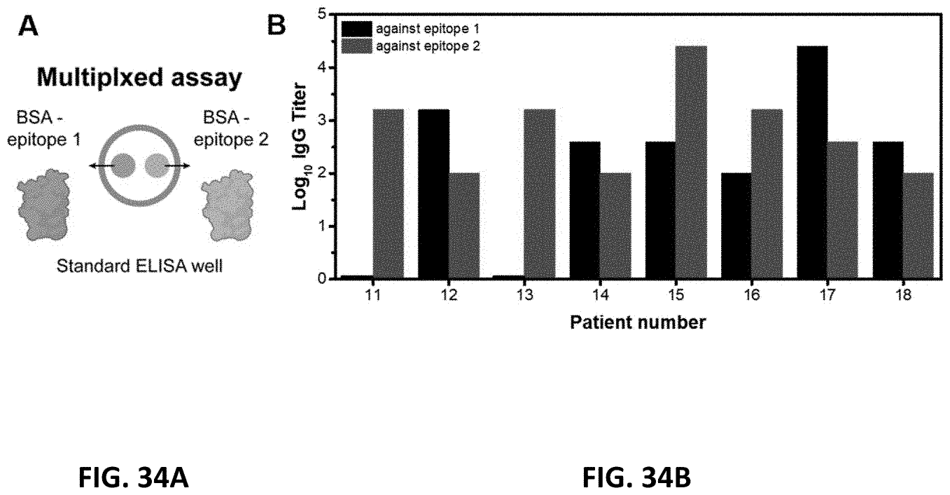

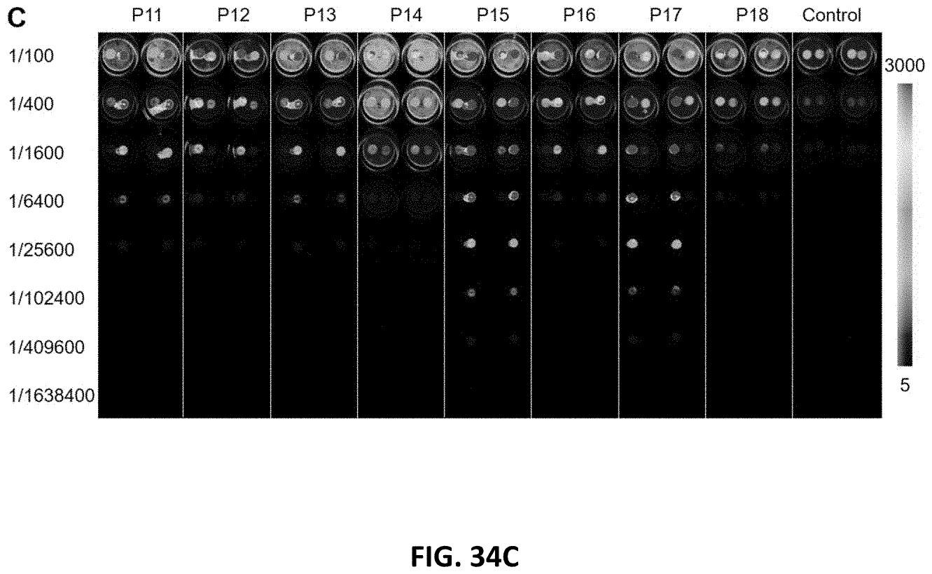

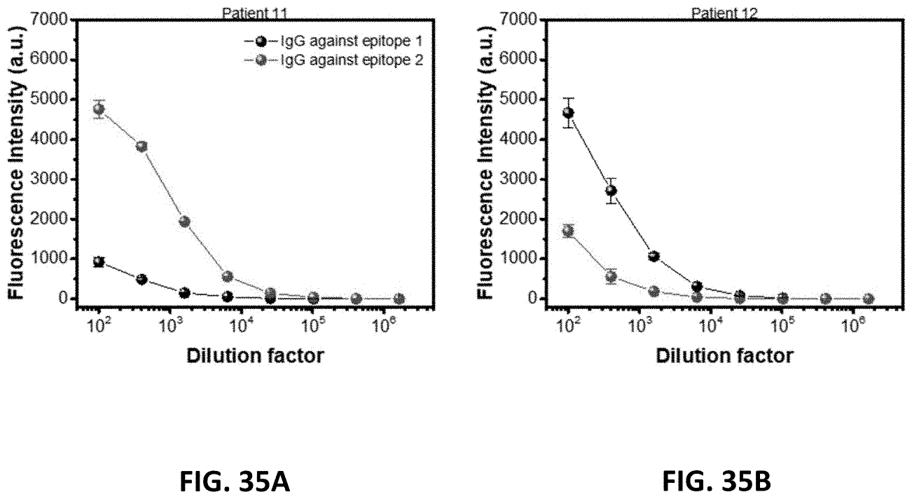

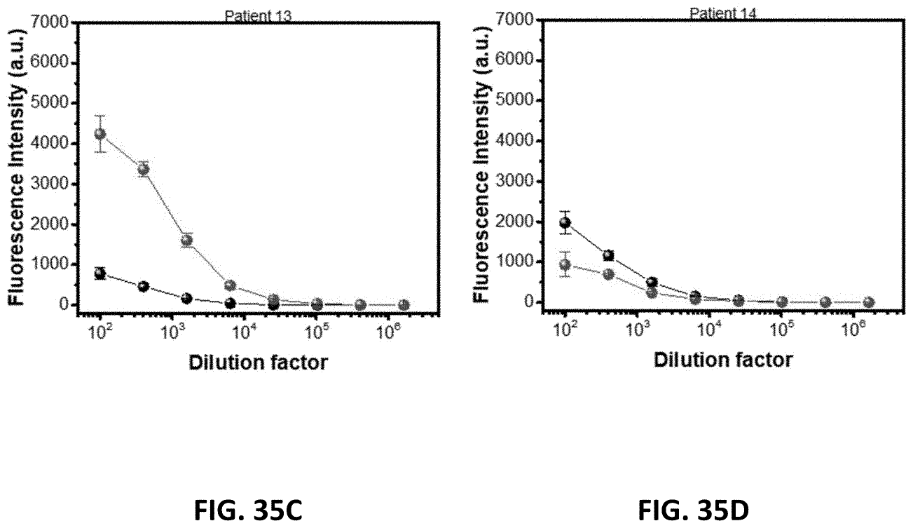

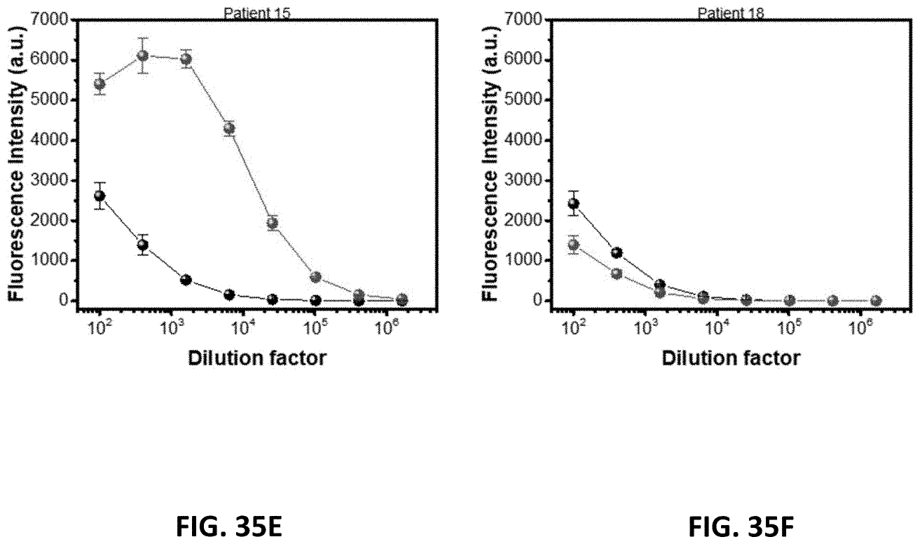

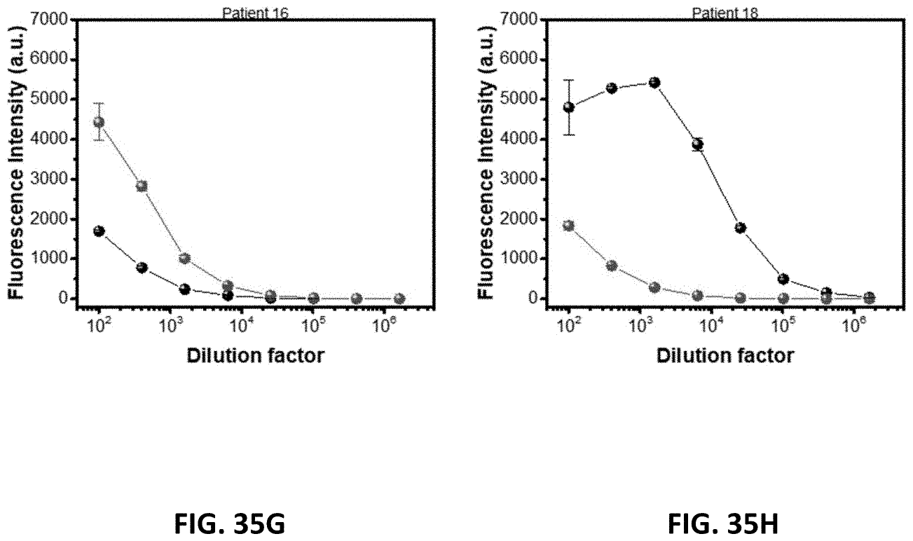

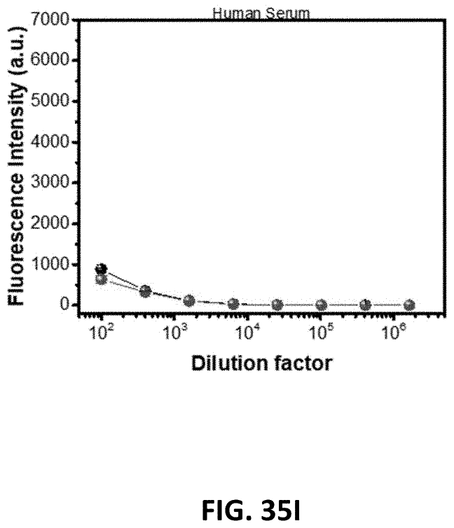

In order to achieve multiplexed detection of epitope specific antibody, a spatial multiplexed dot blot assay was realized by spotting a 2 μl droplet of the two types of BSA-peptide conjugates within one well separately as the capture elements (FIG. 34A). To investigate the feasibility of employing plasmonic-fluor in this multiplexed assay, titer of epitope specific IgG was measured in eight convalescent patient samples with serial dilution. Fluorescence signals of the two BSA-peptide dots demonstrate the expected dilution-dependence, indicating the existence of epitope-specific IgG (FIGS. 34C and 35(A-I)). The fluorescence signal obtained from the rest of the well represents the amount of possible anti-BSA IgG, which was subtracted as the background from the fluorescence signal from the dots. Notably, the antibody titer calculated from the fluorescence intensity reveals the existence of IgG targeting both epitopes, except patient 11 and 13 where the amount of epitope 1-specific IgG are close to the heathy serum control (FIGS. 34B and 35(A-I)). Different IgG titers also uncover the varying IgG profile targeting these two epitopes and indicates distinct immunity acquired upon SARS-CoV-2 infection from patient-to-patient. This detailed information achieved by epitope specific serology assay aids in precise identification of potential convalescent plasma donors and evaluation of vaccine efficacy in population scale.

In summary, an ultrasensitive SARS-CoV-2 epitope-specific serological test was demonstrated in a spatially-multiplexed manner through plasmonically-enhanced fluoroimmunoassay. Plasmonic-fluor, serving as the ultrabright fluorescence reporter, significantly improved the detection sensitivity compared to conventional fluorophores and enzyme-driven colorimetric assay. Specifically, the LOD of the antibody p-FLISA for SARS-CoV-2 is nearly 100-fold better compared to ELISA, completed in 20 minutes. The ultrasensitive detection of various antibody isotypes and epitope-specific antibodies provides more insightful and detailed information about the immune response after infection. Owing to its high sensitivity and specificity, plasmonically-enhanced epitope specific serology assay demonstrated herein is highly attractive to determine the vaccine efficacy, duration of the immunity, and epidemiological investigation of symptomatic patients and discover asymptomatic individuals. The ultrasensitive serology platform introduced here is easily adapted to other infectious diseases by simply replacing the biodetection elements.

Materials and Methods

Patient samples. Samples utilized were obtained from the Washington University School of Medicine's COVID-19 biorepository, which is supported by: the Barnes-Jewish Hospital Foundation; the Siteman Cancer Center grant P30 CA091842 from the National Cancer Institute of the National Institutes of Health; and the Washington University Institute of Clinical and Translational Sciences grant UL1TR002345 from the National Center for Advancing Translational Sciences (NCATS) of the National Institutes of Health (NIH).

Synthesis of plasmonic fluor. Plasmonic-fluor was synthesized according to a previously reported procedure and prepared by Auragent Bioscience LLC.

Synthesis of AuNRs: For plasmonic-fluor 800, AuNRs (LSPR wavelength ˜760 nm) were prepared through a seed-mediated method. Briefly, to prepare seed solution, 600 μl of 10 mM ice-cold NaBH4 solution (Sigma-Aldrich, 71321) was added to a mixture solution comprised of 250 μl of 10 mM HAuC14 (Sigma-Aldrich, 520918) and 9.75 ml of 100 mM hexadecyltrimethylammonium bromide (CTAB, Sigma-Aldrich, H5882), under vigorous stirring at room temperature. The color change of the mixture solution from yellow to brown indicates the formation of seed crystals and the solution was allowed to age in dark for one hour before further usage. To synthesize the gold nanorods, the growth solution was first prepared through sequential addition of CTAB (100 mM, 38 ml), AgNO3 (10 mM, 0.5 ml) (Sigma-Aldrich, 204390), HAuC14 (10 mM, 2 ml), ascorbic acid (0.1 M, 0.22 ml) (Sigma-Aldrich, A92902) and HCl (1 M, 0.9 ml) (Sigma-Aldrich, H9892). Subsequently, 50-fold diluted seed solution was added into the growth solution and left undisturbed overnight in dark. AuNRs were collected by centrifugation at 6000 rpm to remove the supernatant and redispersed in nanopure water for further use.

Conjugation of Biotin and Cy7.5 dye onto BSA: Bovine serum albumin (BSA) was sequentially conjugated with biotin and Cy7.5 dye via EDC/NHS chemistry. First, 2 mg pf NHS-PEG4-Biotin (Thermo Scientific, 21330) was added into 2.2 ml of 5 mg/ml BSA (Sigma-Aldrich, A7030) in 1×PBS. After reaction for one hour, BSA-Biotin conjugation was purified through a desalting column (Thermo Scientific, 89892, 7000 MWCO). To conjugate BSA with Cy7.5 dye, 100 □l of 1 M potassium phosphate dibasic solution (K2HPO4, Sigma Aldrich, P3786) was added into 1 ml BSA-Biotin solution to raise the pH above 9. Subsequently, 25 μl of 4 mg/ml NHS-Cy7.5 (Lumiprob, 16020) was added to the mixture, followed by two-hour incubation at room temperature. BSA-biotin-Cy7.5 was purified through a desalting column pre-equilibrated with nanopure water.

Synthesis of plasmonic fluor: To prepare plasmonic fluor-800, AuNRs (LSPR wavelength around 760 nm), as nanoantennas, were first coated with a thin layer of polymer to avoid fluorescence quench. Briefly, 5 μl of (3-mercaptopropyl)trimethoxysilane (MPTMS, Sigma-Aldrich, 175617) was added into 5 ml of AuNRs solution (extinction around 2), followed by one hour incubation at 24° C. MPTMS modified AuNRs were collected through centrifugation at 6000 rpm for 10 minutes and redispersed in 1 mM CTAB solution. 2 μl APTMS and 2 μl TMPS were sequentially added into the MPTMS modified AuNRs solution to form the polymer layer. Finally, AuNR-polymer were collected through three centrifugations at 6000 rpm for 10 minutes and concentrated into a final volume of 10 μl.

Next, to coat BSA-Biotin-Cy7.5 conjugate around AuNR-polymer, 1 μl of 20 mg/ml citric acid (Alfa Aesar, 36664) was added into 100 μl 4 mg/ml BSA-biotin-Cy7.5 solution. Concentrated AuNR-polymer were subsequently added into the mixture solution and sonicated for 20 minutes in dark. Coated nanostructures were further collected by centrifugation at 4000 rpm for 5 minutes before incubation with 500 μl of 0.4 mg/ml BSA-Biotin-Cy7.5 at pH 10 nanopure water for at least 3 days in 4° C. The nanostructures were washed through 4 times centrifugation at 6000 rpm for 10 minutes using pH 10 water and redisperse in 1% BSA 1×PBS solution before use.

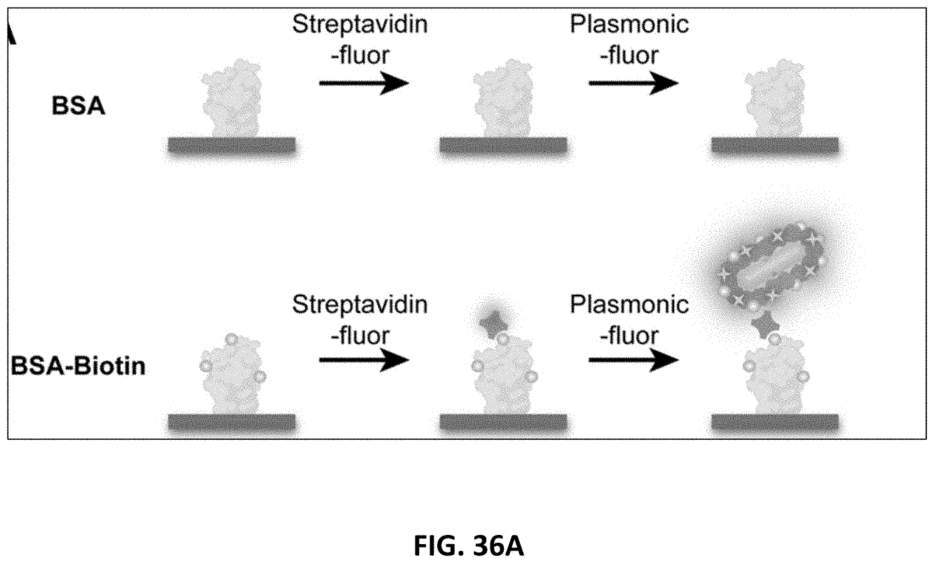

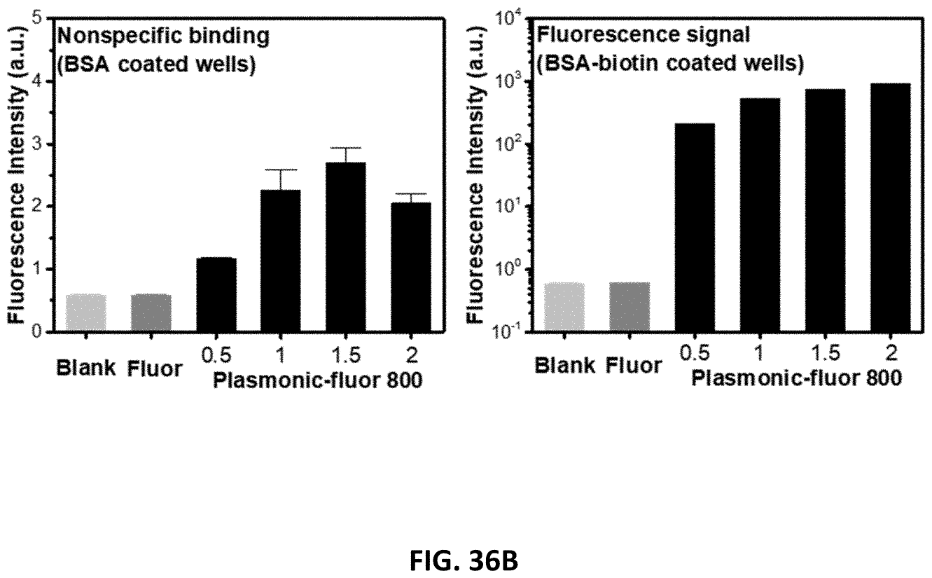

Fluorescence enhancement with plasmonic fluor: The schematic of test procedure was illustrated in FIG. 36(A-B). Specifically, 100 μl 50 ng/ml BSA-biotin was first incubated with 96 well plate for 10 minutes. The plate was subsequently washed by 1×PBS with 0.05% Tween-20 (Sigma Aldrich, P2287) (PBST) and blocked with 3% BSA 1×PBS solution for one hour. Streptavidin-CW800 (1 μg/ml) was added and incubated for 10 minutes, followed by three times washing with PB ST. The plate was further incubated with 76 pM plasmonic fluor-800 in 1% BSA solution. After washing, fluorescence signal before and after incubation of plasmonic fluor were recorded using LI-COR CLx fluorescence scanner with following parameters: channel: 800, laser power: L2, resolution: 21 μm, height: 4 mm.

Conjugation of SARS-CoV-2 epitopes on BSA. Peptides with the sequence of epitopes were ordered from GenScript. To conjugate peptide with BSA, an amino acid spacer, cysteine-glycine-glycine-glycine (CGGG), was designed to be the end group at the N-terminal. The complete sequence is shown below:

Peptide 1:
(SEQ ID NO. 19)
CGGGTESNKKFLPFQQFGRDIA
Peptide 2:
(SEQ ID NO. 20)
CGGGPSKPSKRSFIEDLLFNKV

The obtained peptides were conjugated with BSA through sulfosuccinimidyl-4-(N-maleimidomethyl) cyclohexane-1-carboxylate (Sulfo-SMCC, Thermo Scientific, A39268). First, to conjugate linkers with BSA, sulfo-NHS esters from the linker were reacted with amine groups on BSA. Specifically, 0.5 mg sulfo-SMCC was added to 1 ml of BSA (Sigma-Aldrich, A7030) solution (3 mg/ml in 1×PBS, 1.2 mM EDTA, pH 7.2) and incubate for 30 minutes at room temperature. Conjugated BSA were purified through the desalting column (7000 MWCO, Thermo Scientific, 89890) pre-equilibrated with 1×PBS, 1.2 mM EDTA. To conjugate the peptides, maleimide groups from conjugated BSA were reacted with sulfhydryl groups from peptides through Michael addition. 2 mg peptides were added to 500 μl BSA conjugate solution and incubated for 60 minutes at room temperature. The BSA-peptide was subsequently purified using a desalting column pre-equilibrated with 1×PBS.

Enzyme-linked immunosorbent assay for SARS-CoV-2 antibody detection (within 20 minutes). The detailed information of antigens, targeting antibody and secondary antibody in various types of antibody detection assay are listed in Table 1.

TABLE 1
The pairs of antigen and secondary antibody employed for the detection of
different targeting antibodies in convalescent plasm or standard samples.

| Antigens | Antibodies to be detected | Secondary antibody |
|---|---|---|
| Recombinant SARS-CoV-2 | IgG against N protein in | Biotinylated rabbit anti- |
| nucleocapsid protein | patient samples | human IgG antibodies |
| (Ray Biotech, 230-01104) | or rabbit anti-SARS-CoV-2 | (Rockland, 609-4617) |
| | nucleocapsid IgG | |
| | (Raybiotech, 130-10760) | |
| | IgM against RBD domain in | Biotinylated rabbit anti- |
| | patient samples | human IgM antibodies |
| | | (Rockland, 609-4131) |
| | IgA against RBD domain in | Biotinylated rabbit anti- |
| | patient samples | human IgA antibodies |
| | | (Rockland, 609-4606) |
| Recombinant Spike protein | IgG against S1 protein in | Biotinylated rabbit anti- |
| subunit 1 | patient samples | human IgG antibodies |
| (Ray Biotech, 230-011010) | | (Rockland, 609-4617) |
| | IgM against RBD domain in | Biotinylated rabbit anti- |
| | patient samples | human IgM antibodies |
| | | (Rockland, 609-4131) |
| | IgA against RBD domain in | Biotinylated rabbit anti- |
| | patient samples | human IgA antibodies |
| | | (Rockland, 609-4606) |
| Recombinant Spike protein | IgG against S2 subunit in | Biotinylated rabbit anti- |
| subunit 2 | patient samples | human IgG antibodies |
| (Ray Biotech, 230-01103) | | (Rockland, 609-4617) |
| | IgM against RBD domain in | Biotinylated rabbit anti- |
| | patient samples | human IgM antibodies |
| | | (Rockland, 609-4131) |
| | IgA against RBD domain in | Biotinylated rabbit anti- |
| | patient samples | human IgA antibodies |
| | | (Rockland, 609-4606) |
| Recombinant Spike protein | IgG against RBD domain in | Biotinylated rabbit anti- |
| receptor binding domain | patient samples | human IgG antibodies |
| (Ray Biotech, 230-01102) | or rabbit anti-SARS-CoV-2 | (Rockland, 609-4617) |
| | RBD IgG | |
| | (Raybiotech, 130-10759) | |
| | IgM against RBD domain in | Biotinylated rabbit anti- |
| | patient samples | human IgM antibodies |
| | | (Rockland, 609-4131) |
| | IgA against RBD domain in | Biotinylated rabbit anti- |
| | patient samples | human IgA antibodies |
| | | (Rockland, 609-4606) |
| BSA-peptide conjugates | Epitope specific IgG in | Biotinylated rabbit anti- |
| | patient samples | human IgG antibodies |
| | | (Rockland, 609-4617) |
| | Epitope specific IgM in | Biotinylated rabbit anti- |
| | patient samples | human IgM antibodies |
| | | (Rockland, 609-4131) |
| | Epitope specific IgA in | Biotinylated rabbit anti- |
| | patient samples | human IgA antibodies |
| | | (Rockland, 609-4606) |
| | Epitope specific IgA1 in | Biotinylated mouse anti- |
| | patient samples | human IgA1 antibodies |
| | | (Southern Biotech, 9130-08) |

Generally, eight-well high binding polystyrene ELISA strips (Thermo Scientific, 15031) were pre-coated with 100 μl of antigen solution at a concentration of 2 pg/ml in PBS overnight at 4° C. The wells were washed with PBST and blocked with 350 μl of 3% BSA for 1 hour at room temperature followed. After washing, wells were incubated with commercialized standard samples or serial diluted patient plasma samples in PBST for 5 minutes, followed by washing and incubation of secondary antibody for 5 minutes. Streptavidin-horseradish peroxidase (HRP) (R&D Systems, 893975) were subsequently incubated for 5 minutes. 100 μl of substrate solution (1:1 mixture of color reagent A (H₂O₂) and color reagent B (tetramethylbenzidine)) (R&D Systems, DY999) was incubated and stopped by 50 μl 2 N Sulfuric acid (R&D Systems, DY994) after 5 minutes. Optical density of each well was measured using microtiter plate reader set at 450 nm.

Fluorescence or plasmonic fluor-enhanced immunosorbent assay for SARS-CoV-2 antibody detection (within 20 minutes). The FLISA were implemented using the similar approach as the standard enzymatic immunoassay, except that conventional enzyme mediated reporters were replaced by streptavidin-CW800 (LICOR, 926-32230). In case of p-FLISA, the wells were further washed with PBST for three times and 100 μl plasmonic fluor were subsequently added and incubated for 5 minutes. After washing, the wells were imaged using LI-COR CLx fluorescence imager with the following scanning parameters: laser power ˜L2; resolution ˜21 μm; channel 800; height 4 mm. For the detection of epitope specific antibody, the fluorescence intensity of albumin specific antibody in each plasma sample was subtracted as background to achieve the pristine intensity of SARS-CoV-2 specific antibody.

Plasmonic fluor-enhanced multiplexed detection of epitope specific SARS-CoV-2 antibody. The multiplex detection was achieved by spatial blotting of different BSA-peptide conjugates within the same well of microtiter plate. Specifically, a 2-μl droplet of 4 μg/ml BSA-peptide 1 conjugates in 1×PBS with 10% glycerol were carefully blotted on the left part of the well, followed by another droplet of BSA-peptide 2 conjugates on the right. The microtiter plate was then sealed and incubated in 4° C. overnight, followed by blocking with 300 μl of reagent diluent (1×PBS containing 3% BSA, 0.2 μm filtered). The remaining steps were same as indicated above. For the detection of epitope specific antibody, the fluorescence intensity of albumin specific antibody in each plasma sample (intensity in the rest part of the well) was subtracted as background to achieve the pristine intensity of SARS-CoV-2 specific antibody. To determine the titer of epitope specific antibody in each sample, a cut-off value equals to mean fluorescence signal acquired from healthy control plus three times of standard deviation.

Material characterization. TEM images were obtained using a JEOL JEM-2100F field emission instrument. To prepare the TEM sample, a drop of aqueous solution was dried on a hydrophilic carbon-coated grid. SEM images were obtained using a FEI Nova 2300 field-emission SEM at an accelerate voltage of 10 KV. The extinction spectra of plasmonic nanoparticles were obtained using Shimadzu UV-1800 spectrophotometer. Fluorescence mappings were obtained using LI-COR Odyssey CLx imaging system.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An assay for detecting at least one of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.

2. The assay of claim 1, wherein the SARS-CoV-2 specific antibody is selected from IgG and IgM.

3. The assay of claim 1, wherein the SARS-COV-2 specific immunoglobulin is IgA immunoglobulin that recognizes at least one of SARS-CoV-2 nucleocapsid protein, SARS-CoV-2 Spike protein 1, SARS-CoV-2 Spike protein 2, and Receptor Binding Domain of SARS-CoV-2 Spike protein 1.

4. The assay of claim 1, wherein the assay comprises at least one of an immunomicroarray, an enzyme-linked immunoabsorbent assay (ELISA), a fluorescence linked immunosorbent assay (FLISA), bead-based fluoroimmunoassays, and flow cytometry.

5. The assay of claim 4, wherein the monoclonal response to single linear neutralizing epitopes within SARS-CoV-2 spike protein comprises a single well ELISA assay.

6. The assay of claim 4, wherein the monoclonal response to single linear neutralizing epitopes within SARS-CoV-2 spike protein comprises a multiplexed well ELISA assay.

7. The assay of claim 1, wherein the assay is an ultrafast assay.

8. The assay of claim 1, further comprising biotin and streptavidin.

9. The assay of claim 1, wherein the assay is a dual-modal colorimetric and fluorescence assay.

10. A multiplexed lateral flow assay for detection of two or more of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.

11. The multiplexed lateral flow assay of claim 10, wherein the SARS-CoV-2 specific antibody is selected from IgG, IgM, and at least one monoclonal neutralizing antibody.

12. The multiplexed lateral flow assay of claim 10, wherein the specific antibody comprises autoantibodies.

13. The multiplexed lateral flow assay of claim 10, wherein the SARS-COV-2 specific immunoglobulin is IgA immunoglobulin that recognizes at least one of SARS-CoV-2 nucleocapsid protein, SARS-CoV-2 Spike protein 1, SARS-CoV-2 Spike protein 2, and Receptor Binding Domain of SARS-CoV-2 Spike protein 1.

14. The multiplexed lateral flow assay of claim 10, wherein the assay comprises at least one of an immunomicroarray, an enzyme-linked immunoabsorbent assay (ELISA), a fluorescence linked immunosorbent assay (FLISA), bead-based fluoroimmunoassays, and flow cytometry.

15. The multiplexed lateral flow assay of claim 10, wherein the monoclonal response to single linear neutralizing epitopes within SARS-CoV-2 spike protein comprises a single well ELISA assay.

16. The multiplexed lateral flow assay of claim 15, wherein the monoclonal response to single linear neutralizing epitopes within SARS-CoV-2 spike protein comprises a multiplexed well ELISA assay.

17. The multiplexed lateral flow assay of claim 10, wherein the assay is an ultrafast assay.

18. The multiplexed lateral flow assay of claim 10, further comprising biotin and streptavidin.

19. The multiplexed lateral flow assay of claim 10, wherein the assay is a dual-modal colorimetric and fluorescence assay.

20. A method for amplification of a polyclonal response to SARS-CoV-2 infection, the method comprising performing an assay for detecting at least one of a SARS-CoV-2 specific antibody, a SARS-COV-2 specific immunoglobulin, and a monoclonal response to single linear neutralizing epitopes within SARS-CoV-2 spike protein, wherein the assay comprises a plasmonic-fluor.
